(19) United States
(12) Patent Application Publication
(10) Pub. No.: US 2018/0185459 A1
(43) Pub. Date: Jul. 5, 2018

(54) MODULATION OF FACTOR Xa INHIBITOR-MEDIATED BLOOD LOSS BY PARTIAL AND TRANSIENT ADMINISTRATION OF ANTIDOTE

(71) Applicant: Portola Pharmaceuticals, Inc., South San Francisco, CA (US)

(72) Inventors: Stanley J. Hollenbach, South San Francisco, CA (US); Genmin Lu, Burlingame, CA (US); Uma Sinha, San Francisco, CA (US)

(21) Appl. No.: 15/902,886

(22) Filed: Feb. 22, 2018

Related U.S. Application Data
(63) Continuation of application No. 14/378,440, filed on Aug. 13, 2014, now abandoned.
(60) Provisional application No. 61/599,821, filed on Feb. 16, 2012.
(60) Provisional application No. 61/682,615, filed on Aug. 13, 2012.
(60) Provisional application No. 61/734,269, filed on Dec. 6, 2012.
(60) Provisional application No. 61/756,842, filed on Jan. 25, 2013.
(60) Provisional application No. 61/759,571, filed on Feb. 1, 2013.

(51) Int. Cl. A61K 38/48; C12N 9/64

(52) U.S. Cl. CPC A61K 38/4846; C12Y 304/21006; C12N 9/6432

(57) ABSTRACT

The present disclosure provides unit dose formulations and methods to reduce, stop or prevent bleeding in a patient undergoing anticoagulant therapy with a factor Xa inhibitor. The methods entail at least partial neutralization of the factor Xa inhibitors The unit dose formulations and methods of the present disclosure can be effective even after actual bleeding has initiated.

### CROSS-REFERENCE TO RELATED APPLICATIONS

This application claims the benefit under 35 U.S.C. § 119(e) to U.S. Provisional Applications Ser. Nos. 61/599,821, filed Feb. 16, 2012, 61/682,615, filed Aug. 13, 2012, 61/734,269, filed Dec. 6, 2012, 61/756,842, filed Jan. 25, 2013, and 61/759,571, filed Feb. 1, 2013, the contents of each of which are incorporated by reference in its entirety into the present disclosure.

### FIELD

The present disclosure relates to unit dose formulations of an antidote which can reduce or completely stop factor Xa inhibitor-mediated blood loss by partially or transiently neutralizing the inhibitor. The antidote may be derivatives of factor Xa (fXa) having reduced or lacking intrinsic procoagulant activity but are still capable of binding and/or neutralizing fXa inhibitors, thereby acting as antidotes to such fXa inhibitors. The disclosure is also related to methods of using these unit dose formulations.

### BACKGROUND

Anticoagulants serve a need in the marketplace in treatment or prevention of undesired thrombosis in patients with a tendency to form blood clots, such as those having clotting disorders, confined to periods of immobility or undergoing medical surgeries. One of the major limitations of anticoagulant therapy, however, is the bleeding risk associated with the therapy, and limitations on the ability to rapidly reverse the anticoagulant activity in case of overdosing or if an urgent surgical procedure is required. Thus, specific and effective antidotes to all forms of anticoagulant therapy are highly desirable. For safety considerations, it is also advantageous to have an anticoagulant-antidote pair in the development of new anticoagulant drugs.

One promising form of anticoagulant therapy targets factor Xa (fXa), and in fact, several direct fXa inhibitors are currently in different stages of clinical development for use as anticoagulant therapy. Many of these are small molecules. One direct fXa inhibitor Xarelto™ (rivaroxaban) has been approved for clinical use in the United States, the European Union and Canada for the prevention of venous thromboembolism in orthopedic surgery patients. While these new fXa inhibitors show promise for treatment, specific and effective antidotes are still needed. In case of over-anticoagulation or requirement for surgery in patients treated with these fXa inhibitors, an agent may be required to substantially neutralize the administered fXa inhibitor or inhibitors and restore normal hemostasis.

Currently available antidotes for anticoagulant therapy, such as recombinant factor VIIa (rfVIIa), are mechanistically limited and not specific for reversal of fXa inhibitors and thus improved options for the clinician are highly desirable. In human studies, rfVIIa has been used to reverse the effect of indirect antithrombin III (ATIII)-dependent fXa inhibitors such as fondaparinux and idraparinux (Bijsterveld, NR et al., Circulation, 2002, 106:2550-2554; Bijsterveld, NR et al., British J. of Haematology, 2004(124): 653-658). The mechanism of action of factor VIIa (fVIIa) is to act with tissue factor to convert factor X (fX) present in blood circulation to active fX (fXa) to restore normal hemostasis in patients. This mode of action necessarily dictates that the highest potential concentration of fXa that could be attained to neutralize active site-directed fXa inhibitors is limited by the circulating plasma concentration of fX. Thus the potential of using rfVIIa to reverse the effect of direct fXa inhibitors is mechanistically limited.

Exogenous fXa cannot be administered directly to a subject in a way similar to rfVIIa. Unlike rfVIIa, which has very low procoagulant activity in the absence of its cofactor tissue factor, native fXa is a potent enzyme and has a potential risk of causing thrombosis. Thus, the use of either rfVIIa or active fXa as an antidote to a fXa anticoagulant therapy has disadvantages.

Antidotes employed in the formulations and methods of the disclosure are described in U.S. Patent Application Publication 2009-0098119. This publication, and any publications, patents (e.g., U.S. Pat. No. 8,153,590), patent applications mentioned herein, are hereby incorporated by reference in their entirety.

Notwithstanding the disclosure of antidotes in the above mentioned application, the dosing of the antidote is an important component to ensure patient safety.

### SUMMARY

The present disclosure provides, in one embodiment, a unit dose formulation for neutralizing a factor Xa inhibitor, comprising a pharmaceutically acceptable carrier and from about 25 milligrams to about 95 milligrams of a two-chain polypeptide comprising the amino acid sequence of SEQ ID NO. 3 or a polypeptide having at least 80% sequence identity to SEQ ID NO. 3. In one aspect, the polypeptide having at least 80% sequence identity to SEQ ID NO. 3 is a biological equivalent of SEQ ID NO: 3. A biological equivalent of SEQ ID NO: 3, in some aspects, has reduced procoagulant activity compared to wild-type factor Xa and does not assemble into a prothrombinase complex.

In one aspect, the unit dose formulation contains from about 30 milligrams to about 80 milligrams, or from about 35 milligrams to about 70 milligram, or from about 35 milligrams to about 60 milligrams of the polypeptide.

In another aspect, the unit dose formulation is formulated for administration as a single bolus.

The factor Xa inhibitor, in one aspect, is a direct factor Xa inhibitor, such as but not limited to NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof. In a particular aspect, the direct factor Xa inhibitor is rivaroxaban.

The factor Xa inhibitor, in one aspect, is an indirect factor Xa inhibitor, such as but not limited to fondaparinux, idraparinux, biotinylated idraparinux, enoxaparin, fragmin, tinzaparin, low molecular weight heparin and combinations thereof. In a particular aspect, the indirect factor Xa inhibitor is enoxaparin.

Some embodiments provide methods of using the unit dose formulations of the present disclosure. One embodiment provides a method of selectively binding and inhibiting an exogenously administered direct or indirect factor Xa inhibitor in a subject undergoing anticoagulant therapy with a factor Xa inhibitor comprising administering to the subject an injection of a unit dose formulation of the present disclosure.

Anther embodiment provides a method of preventing, reducing, or ceasing bleeding in a subject undergoing anticoagulant therapy with a direct or indirect factor Xa inhibitor comprising administering to the subject an injection of a unit dose formulation of the present disclosure.

Yet another embodiment provides a method for correcting fXa inhibitor dependent pharmacodynamic or surrogate markers in a patient undergoing anticoagulant therapy with a direct or indirect factor Xa inhibitor comprising administering to the subject an injection of a unit dose formulation of the present disclosure. In one aspect, the pharmacodynamic or surrogate marker is selected from the group consisting of INR, PT, aPTT, ACT, anti-fXa units, and thrombin generation.

Also provided, in one embodiment, is a method of reducing or ceasing bleeding in a subject undergoing an anticoagulant therapy with a direct factor Xa inhibitor, comprising administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reaches a circulating molar concentration in the subject that is less than about 95% of the circulating molar concentration of the direct factor Xa inhibitor. Alternatively, the polypeptide reaches a circulating molar concentration in the subject that is less than about 70% of the circulating molar concentration of the direct factor Xa inhibitor.

Still, one embodiment of the present disclosure provides a method of reducing or ceasing bleeding in a subject undergoing an anticoagulant therapy with an indirect factor Xa inhibitor, comprising administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reduces from about 20% to about 95% of the level of an anti-fXa pharmacodynamic marker of the indirect factor Xa inhibitor. In some aspects, the polypeptide reduces from about 25% to about 80%, or from about 35% to about 65% of the level of the anti-fXa pharmacodynamic marker. In one aspect, the administration is via a single bolus.

Methods of reducing or ceasing bleeding in a subject having received an anticoagulant therapy with a factor Xa inhibitor and experiencing clinically relevant bleeding are also provided. In one aspect, the methods entail administering to the subject a therapeutically effective amount of a formulation comprising a two-chain polypeptide comprising the amino acid sequence of SEQ ID NO. 3 or a polypeptide having at least 80% sequence identity to SEQ ID NO. 3. In one aspect, the administration comprises an injection of the formulation. In another aspect, the administration is at least about 5 minutes, or alternatively at least about 10 or 15 minutes, after the blood loss has initiated. In some aspects, the polypeptide neutralizes between about 20% and about 95%, or between about 35% and about 65% of the factor Xa inhibitor.

Still another embodiment of the present disclosure provides a method of controllably neutralizing the anticoagulant activity of a factor Xa inhibitor in a subject undergoing an anticoagulant therapy with the factor Xa inhibitor, comprising measuring the level of an anti-fXa pharmacodynamic marker of the factor Xa inhibitor, and administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reduces from about 20% to about 95% of the level of the anti-fXa pharmacodynamic marker. In some aspects, the polypeptide reduces from about 35% to about 65% of the level of the anti-fXa pharmacodynamic marker.

### BRIEF DESCRIPTION OF THE DRAWINGS

FIG. 1A-B show the effect of a single bolus of r-Antidote administration in neutralizing the anticoagulant activity of rivaroxaban, a direct factor Xa inhibitor, and reducing rivaroxaban-induced bleeding. FIG. 1A is a graph showing the increase in the pharmacodynamic (PD) marker, anti-fXa activity, due to rivaroxaban (Riva) anticoagulation, and the reversal of the PD marker by r-Antidote (AD). The anti-fXa activity was normalized to aspirin (ASA)+Riva group. The vehicle- and ASA-treated groups were below the limit of quantitation. FIG. 1B shows the increase in blood loss due to Riva and/or ASA treatment, and the reduction of blood loss by r-Antidote. The blood loss amount was expressed as mean ±SEM (n=8/group).

FIG. 2A-C show that, despite the short half life of the r-Antidote in rats and the rebound of anticoagulant activity of the indirect factor Xa inhibitor, enoxaparin, a single bolus dose of r-Antidote is as effective as a bolus+infusion administration of the r-Antidote. FIG. 2A presents the cumulative blood loss curves in each annotated treatment, showing that r-Antidote bolus (prophylactic administration) completely inhibited blood loss due to anticoagulation by an indirect factor Xa inhibitor, enoxaparin, even in the case of partial reversal of anti-fXa activity (shown in FIG. 2B) in a rat blood loss model. FIG. 2B shows that the PD marker, anti-fXa activity, was reversed and sustained by a bolus plus infusion regimen, but bolus-alone administration was sufficient to reduce blood loss, even though the PD marker (anti-fXa activity) increased over time to the level of enoxaparin alone. FIG. 2C presents ELISA results showing that the plasma concentration of the r-Antidote decreased, starting at 15 minutes following the bolus administration, to a greater extent in rats receiving the bolus only administration than in those receiving both bolus and infusion administrations.

FIG. 3A-D present data to show that partial and transient reversal of the anticoagulant activity of enoxaparin was effective in reducing or stopping enoxaparin-induced bleeding. These figures also demonstrate the effectiveness of the treatment even after the bleeding was already occurring. FIG. 3A presents cumulative blood loss data to show that a single bolus dose of r-Antidote, at five minutes after tail transection, was effective in stopping the enoxaparin-induced bleeding. FIG. 3B summarizes the cumulative blood loss at 45 minutes after tail transection and FIG. 3D presents the time course for each treatment. In this experiment, the bolus dose of the r-Antidote was administered 10 minutes after the tail transection. Among the four different doses of r-Antidote used, 1, 2 and 4 mg bolus doses were all effective in significantly reducing blood loss. FIG. 3C presents the measured anti-fXa activity in the animals. It is shown that a 1 mg bolus dose of r-Antidote resulted in about 35% reversal of the PD marker (anti-fXa activity) and yet was adequate to reduce bleeding effectively.

FIG. 4A-B demonstrates the in vitro reversal of enoxaparin anticoagulation thrombin generation and the anti-fXa activity. FIG. 4A: effect of r-Antidote on enoxaparin mediated inhibition of thrombin generation in human plasma; FIG. 4B: r-Antidote dose-dependently reversed the anti-fXa activity of therapeutic concentration of enoxaparin in rat and human plasma.

FIG. 5 shows reduction of blood loss by r-Antidote (PRT) in rivaroxaban-anticoagulated rabbits. Administration of rivaroxaban increased mean blood loss >3-fold compared with vehicle-treated group (blood loss: vehicle group 6.6±2.1 grams, rivaroxaban group 22.7±9.3 grams). Administration of PRT, rfVIIa or PCC (prothrombin complex concentrate) alone did not affect blood loss in the absence of rivaroxaban. Prophylactic reversal of anticoagulation in rivaroxaban treated rabbits with r-Antidote (75 mg/rabbit) significantly reduced blood loss by 76%, p<0.01 (Riva+PRT vs. Riva alone). Administration of rfVIIa (150 μg/kg NovoSeven) or PCC (60 IU/kg Bebulin) in rivaroxaban anticoagulated rabbits had no effect on blood loss. Average rivaroxaban plasma concentration for the riva alone, riva+PRT, riva+rfVIIa and riva+PCC dose groups just prior to treatment was 1.2, 1.6, 1.0 and 0.8 μM, respectively.

FIG. 6 presents a chart that summarizes the reduction of blood loss by r-Antidote (PRT) in rivaroxaban-anticoagulated rabbits.

FIG. 7 shows the effect of r-Antidote (PRT) administration on reducing plasma free fraction of rivaroxaban. Free rivaroxaban plasma concentration (not bound to plasma protein or r-Antidote) was similar for all groups prior to r-Antidote treatment at the 30 minute time point. The unbound fraction of rivaroxaban was significantly reduced by 94% (35 minute) and 70% (50 minute) only in r-Antidote-treated group, *p<0.001(Riva+PRT vs. Riva).

FIG. 8 shows the effect of r-Antidote administration on reduction in anti-fXa activity due to rivaroxaban anticoagulation. Only with administration of r-Antidote was there a significant (*p<0.05) reduction in anti-fXa activity compared to pre-treatment measurements.

FIG. 9A-C show that r-antidote reverses the inhibitory activity of three direct fXa inhibitors. FIG. 9A shows the dose dependent reversal of betrixaban, rivaroxaban and apixaban inhibition in an fXa enzyme assay. Residual fXa activity was determined after incubation of r-antidote (0-250 nM) at room temperature (RT) for 30 min with different concentrations of the inhibitors: 0 (□), 2.5(Δ), 5.0(⋄), and 7.5 nM (◯). The affinity of r-antidote to inhibitor (K_{d,antidote}) was calculated with Dynafit using reported K_{i,fXa} for each inhibitor. The lines represent the best fittings; Kd and Ki values are listed. mOD min⁻¹=miliOptical Density per minute. FIG. 9B shows the reversal of prolongation of prothrombin time (PT) produced by rivaroxaban in human plasma. Rivaroxaban (1 μM) was incubated with different concentrations of r-antidote at RT for 30 min before initiation of assay. Grey bar: Control plasma alone (PPP), Filled bar: rivaroxaban or rivaroxaban+r-antidote, Open bar: Control plasma+r-antidote. FIG. 9C shows that r-antidote lacks pro- or anticoagulant activity in thrombin generation assay. Human plasma with increasing concentrations of r-Antidote (□) or EGR-Xa (◯) was incubated for 30 min at RT. Thrombin generation was initiated by addition of Ca^{2|} and tissue factor (TF). Cleavage of thrombin specific substrate Z-GGR-AMC (RFU=Relative Fluorescence Units) was measured at 37° C. for 10 min.

FIG. 10A-C show that r-antidote reverses markers of anticoagulation in fXa inhibitor treated rats as evidenced by a sustained reversal of whole blood international normalized ratio (INR). In FIG. 10A, rats were infused with rivaroxaban (0.25 mg/kg/hr) or vehicle for 30 min followed by treatment with either vehicle or r-antidote by IV bolus (4 mg/rat) over 5 min plus infusion (4 mg/hr) up to 90 min. Whole blood INR (mean±SD, n=4/group) was measured by Hemochron Jr microcoagulation system. Treated groups: (◯) vehicle+vehicle; (□) rivaroxaban+vehicle; (Δ) rivaroxaban+r-Antidote. **P-value (r-Antidote vs. rivaroxaban)<0.01. In FIG. 10B, rats were infused with betrixaban (1 mg/kg/hr) or vehicle followed by treatment with either vehicle or r-Antidote by IV bolus (6 mg/rat) over 5 min plus infusion (9 mg/hr) up to 90 min. Whole blood INR (mean±SD, n=4/group) is presented. Treated groups: (◯) vehicle+vehicle; (□) betrixaban+vehicle; (Δ) betrixaban+r-Antidote. *P-value (r-Antidote vs. betrixaban)<0.02. In FIG. 10C, rats were infused with apixaban (0.5 mg/kg/hr) or vehicle followed by treatment with either vehicle or r-Antidote by IV bolus (6 mg/rat) over 5 min plus infusion (6 mg/hr) up to 90 min. Whole blood INR (mean±SD, n=5-6/group) is presented. Treated groups: (◯) vehicle+vehicle; (□) apixaban+vehicle; (Δ) apixaban+r-Antidote. *P-value (r-Antidote vs. apixaban)<0.01. INR =international normalized ratio; min=minute.

FIG. 11 shows the change of total versus unbound rivaroxaban concentrations upon r-Antidote treatment. Total rivaroxaban plasma concentration at different time points from FIG. 10A was determined by LC-MS (solid line). Fraction of free inhibitor not bound to plasma or r-Antidote protein was first isolated by centrifugation with a 10 kDa cut off Centricon filter and quantified by LC-MS (dashed line) following treatment with rivaroxaban+vehicle (n=4) (◯) or rivaroxaban+r-Antidote (n=5) (∇).

FIG. 12 shows that r-Antidote reduces blood loss in rivaroxaban treated mice. Mice (n=5-8/group) were first dosed orally with vehicle or aspirin at ˜100 mg/kg/day for 5 days. Rivaroxaban (50 mg/kg) or vehicle (5 mL/kg) was then dosed orally. Two hours after rivaroxaban administration, animals were treated with r-Antidote (0.96 mg/mouse, IV bolus) or vehicle (200 μL/mouse). Blood loss was measured for 15 min following treatment with vehicle+vehicle+vehicle (Treat-I), vehicle+rivaroxaban+vehicle (Treat-II), ASA+vehicle+vehicle (Treat-III), ASA+rivaroxaban+vehicle (Treat-IV), and ASA+rivaroxaban+r-Antidote (Treat-V). •P-value (vs. Treat-I or Treat-III)=ns; ***P-value (vs. Treat-III or Treat-V)≤0.001. NS=non-significant

FIG. 13 shows the mitigation of blood loss due to rivaroxaban-induced anticoagulation with r-Antidote in a rabbit liver laceration model. Rivaroxaban (1 mg/kg) or vehicle was dosed by IV bolus over 2 minutes via marginal ear vein catheter. After 30 minutes, r-Antidote or vehicle was administered as an IV bolus injection over 5 minutes followed by laceration of two liver lobes with a scalpel blade and lost blood was collected on pre-weighed gauze over 15 minutes. Riva=Rivaroxaban; g=gram

FIG. 14A-B shows that r-Antidote dose-dependently reverses inhibitory activity of indirect fXa inhibitors LMWH (enoxaparin) and fondaparinux. FIG. 14A shows the reversal of enoxaparin (1 IU/mL) in human (◯) or rat (⋄) plasma. FIG. 14B shows the reversal of fondaparinux (2 μg/mL) in human (◯) or rat (⋄) plasma. Standard curve of anti-fXa activity was generated by known concentrations of enoxaparin (IU/mL) or fondaparinux (μg/mL), and normalized to the starting value of each anticoagulant in the absence of r-Antidote.

FIG. 15A-B shows that r-Antidote reverses the inhibition of fXa by ATIII-fondaparinux. FIG. 15A shows the reversal of fXa activity was measured in the presence of increasing concentrations of r-Antidote. The solid lines represent the non-linear fitting of the experimental data with a single exponential decay function. Using this fit, an apparent association rate constant (k_{obs}) for the reaction were calculated with different concentrations of r-Antidote. In the absence of fondaparinux, r-Antidote had minimal effect on the inhibition of fXa by ATIII (data not shown). FIG. 15B shows the effect of r-Antidote on the kinetic parameter kiₐₚₚ, the apparent second order rate constant for fXa inhibition by the ATIII-fondaparinux complex or ATIII alone. kiapp was defined as k_{obs} divided by the total concentration of ATIII-fondaparinux complex (equal to the limiting reagent,100 nM fondaparinux) or ATIII in the reaction mixture.

FIG. 16A-C shows the reversal of ATIII-dependent fXa Inhibitors by r-Antidote in rat tail-transection model. FIG. 16A shows the dose-titration of r-Antidote for mitigation of blood loss due to enoxaparin-induced anticoagulation. Enoxaparin (4.5 mg/kg) or vehicle was dosed by IV bolus over 5 min, followed by treatment with vehicle or two different doses of r-Antidote (2 mg/rat bolus plus 2 mg/hr infusion and 4 mg/rat bolus plus 4 mg/hr infusion) starting at 10 min. Blood loss was measured for 15 min as described under Methods section. FIG. 16B shows the mitigation of blood loss due to fondaparinux-induced anticoagulation with r-Antidote. Fondaparinux (25 mg/kg) or vehicle was dosed as IV bolus over 5 min followed by treatment with r-Antidote (6 mg/rat bolus plus 6 mg/hr infusion) or vehicle starting at 10 min. Blood loss was measured as in FIG. 16A. Protamine was dosed as an IV bolus only (0.9 mg/rat). FIG. 16C shows the reversal of fondaparinux anti-fXa activity. The anti-fXa activity in fondaparinux anticoagulated rats (FIG. 16B) at different time points are presented for Treat-I: (not shown in anti-fXa), Treat-II (□), Treat-III (⋄), and Treat-IV (∇). Anti-fXa activity was normalized to the activity of Treat-II at 5 min time point. ***P-value (Treat-III vs. Treat-II)≤0.0001.

FIG. 17 shows the anti-fXa activity of rivaroxaban (50 ng/ml Riva) in the presence of various doses of r-Antidote.

### DETAILED DESCRIPTION

### I. Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

The term “protein” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, amino, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Single letter and three letter abbreviations of the naturally occurring amino acids are listed below. A peptide of three or more amino acids is commonly called an oligopeptide if the peptide chain is short. If the peptide chain is long, the peptide is commonly called a polypeptide or a protein.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” which when referring to a reference protein, antibody, polypeptide or nucleic acid, intends those having minimal homology while still maintaining desired structure or functionality. In an alternative embodiment, the term “biological equivalent of” a polynucleotide refers to one that hybridizes under stringent conditions to the reference polynucleotide or its complement. Unless specifically recited herein, it is contemplated that any polynucleotide, polypeptide or protein mentioned herein also includes equivalents thereof. For example, an equivalent intends at least about 80% homology or sequence identity and alternatively, at least about 85%, or alternatively at least about 90%, or alternatively at least about 95%, or alternatively 98% percent homology or sequence identity and exhibits substantially equivalent biological activity to the reference protein, polypeptide or nucleic acid.

“Hybridization” refers to hybridization reactions that can be performed under conditions of different “stringency”. Conditions that increase the stringency of a hybridization reaction are widely known and published in the art: see, for example, Sambrook, et al., infra. Examples of relevant conditions include (in order of increasing stringency): incubation temperatures of 25° C., 37° C., 50° C., and 68° C.; buffer concentrations of 10 X SSC, 6 X SSC, 1 X SSC, 0.1 X SSC (where SSC is 0.15 M NaCl and 15 mM citrate buffer) and their equivalent using other buffer systems; formamide concentrations of 0%, 25%, 50%, and 75%; incubation times from 5 minutes to 24 hours and washes of increasing duration, increasing frequency, or decreasing buffer concentrations.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank +EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

The term “factor Xa inhibitors” or “inhibitors of factor Xa” refers to compounds that can inhibit, either directly or indirectly, the coagulation factor Xa's activity of catalyzing conversion of prothrombin to thrombin in vitro and/or in vivo. Examples of known fXa inhibitors include, without limitation, edoxaban, fondaparinux, idraparinux, biotinylated idraparinux, enoxaparin, fragmin, NAP-5, rNAPc2, tissue factor pathway inhibitor, DX-9065a (as described in, e.g., Herbert, J. M., et al., J Pharmacol Exp Ther. 1996 276(3):1030-8), YM-60828 (as described in, e.g., Taniuchi, Y., et al., Thromb Haemost. 1998 79(3):543-8), YM-150 (as described in, e.g., Eriksson, B. I. et. al, Blood 2005;106(11), Abstract 1865), apixaban, rivaroxaban, PD-348292 (as described in, e.g., Pipeline Insight: Antithrombotics—Reaching the Untreated Prophylaxis Market, 2007), otamixaban, razaxaban (DPC906), BAY 59-7939 (as described in, e.g., Turpie, A. G., et al., J. Thromb. Haemost. 2005, 3(11):2479-86), edoxaban (as described in, e.g., Hylek E M, Curr Opin Invest Drugs 2007 8(9):778-783), LY517717 (as described in, e.g., Agnelli, G., et al., J. Thromb. Haemost. 2007 5(4):746-53), GSK913893, betrixaban (as described below) and derivatives thereof. Low molecular weight heparin (“LMWH”), such as tinzaparin (Innohep), is also considered a factor Xa inhibitor.

“Direct factor Xa inhibitors” are factor Xa inhibitors that act directly upon factor Xa in the coagulation cascade, without involving antithrombin III (ATIII). Non-limiting examples of direct factor Xa inhibitors include NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

“Indirect factor Xa inhibitors” inhibit the activity of factor Xa relying on the presence of ATIII and do not directly interact with factor Xa. Non-limiting examples of indirect factor Xa inhibitors include fondaparinux, idraparinux, biotinylated idraparinux, enoxaparin, fragmin, tinzaparin (Innohep), low molecular weight heparin and combinations thereof.

“Neutralize,” “reverse,” “correct,” or “counteract” the activity of an inhibitor of fXa or similar phrases refer to inhibiting or blocking the factor Xa inhibitory or anticoagulant function of an fXa inhibitor. Such phrases refer to partial inhibition or blocking of the function, as well as to inhibiting or blocking most or all of fXa inhibitor activity, in vitro and/or in vivo. The neutralization effect can be measured with pharmacodynamic (PD) or surrogate markers. Examples of markers include, but are not limited to, INR (international normalized ratio), PT (prothrombin time), aPTT (activated partial thromboplastin time), ACT (activated clotting time), anti fXa units, thrombin generation (Technothrombin TGA, thromboelastography, CAT (calibrated automated thrombogram)) and the like.

“Factor Xa” or “fXa” or “fXa protein” is a serine protease in the blood coagulation pathway, which is produced from the inactive factor X (fX, SEQ ID NO. 1, Table 1). The nucleotide sequence coding human factor X (“fX”) can be found in GenBank with accession number “NM_000504.” Upon catalytic cleavage of the first 52 residues of the heavy chain, fX is activated to fXa. FXa contains a light chain and a heavy chain. The first 45 amino acid residues (residues 1-45 of SEQ ID NO. 1) of the light chain is called the Gla domain because it contains 11 post-translationally modified γ-carboxyglutamic acid residues (Gla). It also contains a short (6 amino acid residues) aromatic stack sequence (residues 40-45 of SEQ ID NO. 1). Chymotrypsin digestion selectively removes the 1-44 residues resulting in Gla-domainless fXa. The serine protease catalytic domain of fXa locates at the C-terminal heavy chain. The heavy chain of fXa is highly homologous to other serine proteases such as thrombin, trypsin, and activated protein C.

“Native fXa” or “wild-type fXa” refers to the fXa naturally present in plasma or being isolated in its original, unmodified form, which processes the biological activity of activating prothrombin therefore promoting formation of blood clot. The term includes naturally occurring polypeptides isolated from tissue samples as well as recombinantly produced fXa. “Active fXa” refers to fXa having the procoagulant activity of activating prothrombin. “Active fXa” may be a native fXa or modified fXa that retains procoagulant activity.

As used herein, “fXa derivatives” refer to modified fXa proteins that do not compete with fXa in assembling into the prothrombinase complex and have reduced or no procoagulant activities, and yet bind and/or substantially neutralize the anticoagulants, such as fXa inhibitors. “Procoagulant activity” of an fXa protein or fXa derivative, in some aspects, refers to the enzymatic activity that the wild-type active fXa polypeptide carries. Examples of fXa derivatives are provided in U.S. Pat. No. 8,153,590, and PCT publications WO2009/042962 and WO2010/056765, and further provided herein, such as SEQ ID NO: 2 and 3 and biological equivalents thereof.

The “enzymatic activity” of an fXa polypeptide or derivatives thereof refers to the polypeptide's ability to catalyze a biochemical reaction with a substrate through direct interaction with the substrate.

SEQ ID NO: 2 contains 3 mutations relative to the wild type fXa. The first mutation is the deletion of 6-39 aa in the Gla-domain of fX. The second mutation is replacing the activation peptide sequence 143-194 aa with —RKR— (SEQ ID NO: 5). This produces a —RKRRKR— (SEQ ID NO: 4) linker connecting the light chain and the heavy chain. Upon secretion, this linker is cleaved resulting in a two-chain polypeptide, SEQ ID NO: 3 (r-Antidote). The third mutation is mutation of active site residue S379 to an Ala residue. This amino acid substitution corresponds to amino acid 296 and 290 of SEQ ID NOS: 1 and 3, respectively.

The term “r-Antidote” refers to a processed two-chain polypeptide processing product of SEQ ID NO: 2, after cleavage of the linker. This is represented by SEQ ID NO: 3. The present disclosure provides a variety of biological equivalents of r-Antidote (or their precursors, represented by SEQ ID NO: 2), or alternatively polypeptides having certain sequence identity to SEQ ID NO: 3. In one aspect, such biological equivalents retain the structural characteristics of SEQ ID NO: 3, that is, a modified active site and a deleted or modified Gla domain. In another aspect, such biological equivalents retain the functional features of SEQ ID NO: 3, that is, not competing with fXa in assembling into the prothrombinase complex and having reduced or no procoagulant activities.

TABLE 1
Polypeptide Sequence of Inactive Human Factor X (SEQ ID NO: 1)

| | |
|---|---|
| 1 | ANSFLEEMKK GHLERECMEE TCSYEEAREV FEDSDKTNEF WNKYKDGDQC ETSPCQNQGK |
| 61 | CKDGLGEYTC TCLEGFEGKN CELFTRKLCS LDNGDCDQFC HEEQNSVVCS CARGYTLADN |
| 121 | GKACIPTGPY PCGKQTLERR KRSVAQATSS SGEAPDSITW KPYDAADLDP TENPFDLLDF |
| 181 | NQTQPERGDN NLTRIVGGQE CKDGECPWQA LLINEENEGF CGGTILSEFY ILTAAHCLYQ |
| 241 | AKRFKVRVGD RNTEQEEGGE AVHEVEVVIK HNRFTKETYD FDIAVLRLKT PITFRMNVAP |
| 301 | ACLPERDWAE STLMTQKTGI VSGFGRTHEK GRQSTRLKML EVPYVDRNSC KLSSSFIITQ |
| 361 | NMFCAGYDTK QEDACQGDSG GPHVTRFKDT YFVTGIVSWG EGCARKGKYG IYTKVTAFLK |
| 421 | WIDRSMKTRG LPKAKSHAPE VITSSPLK |

TABLE 2
Polypeptide Sequence of the r-Antidote prior to removal of the
-RKRRKR- (SEQ ID NO. 4) linker (SEQ ID NO: 2)
Light Chain

| | |
|---|---|
| 1 | ANSFL F WNKYKDGDQC ETSPCQNQGK |
| 61 | CKDGLGEYTC TCLEGFEGKN CELFTRKLCS LDNGDCDQFC HEEQNSVVCS CARGYTLADN |
| 121 | GKACIPTGPY PCGKQTLER |

Linker

RKRRKR

Heavy Chain

| | |
|---|---|
| 181 | IVGGQE CKDGECPWQA LLINEENEGF CGGTILSEFY ILTAAHCLYQ |
| 241 | AKRFKVRVGD RNTEQEEGGE AVHEVEVVIK HNRFTKETYD FDIAVLRLKT PITFRMNVAP |
| 301 | ACLPERDWAE STLMTQKTGI VSGFGRTHEK GRQSTRLKML EVPYVDRNSC KLSSSFIITQ |
| 361 | NMFCAGYDTK QEDACQGDAG GPHVTRFKDT YFVTGIVSWG EGCARKGKYG IYTKVTAFLK |
| 421 | WIDRSMKTRG LPKAKSHAPE VITSSPLK |

TABLE 3
Polypeptide Sequence ofa Human Factor Xa triple mutant after
removal of the -RKRRKR- (SEQ ID NO. 4) linker (SEQ ID NO: 3)
Light Chain

| | |
|---|---|
| 1 | ANSFL F WNKYKDGDQC ETSPCQNQGK |
| 61 | CKDGLGEYTC TCLEGFEGKN CELFTRKLCS LDNGDCDQFC HEEQNSVVCS CARGYTLADN |
| 121 | GKACIPTGPY PCGKQTLER |

Heavy Chain

| | |
|---|---|
| 181 | IVGGQE CKDGECPWQA LLINEENEGF CGGTILSEFY ILTAAHCLYQ |
| 241 | AKRFKVRVGD RNTEQEEGGE AVHEVEVVIK HNRFTKETYD FDIAVLRLKT PITFRMNVAP |
| 301 | ACLPERDWAE STLMTQKTGI VSGFGRTHEK GRQSTRLKML EVPYVDRNSC KLSSSFIITQ |
| 361 | NMFCAGYDTK QEDACQGDAG GPHVTRFKDT YFVTGIVSWG EGCARKGKYG IYTKVTAFLK |
| 421 | WIDRSMKTRG LPKAKSHAPE VITSSPLK |

The term “active site” refers to the part of an enzyme or antibody where a chemical reaction occurs. A “modified active site” is an active site that has been modified structurally to provide the active site with increased or decreased chemical reactivity or specificity. Examples of active sites include, but are not limited to, the catalytic domain of human factor X comprising the 235-488 amino acid residues, and the catalytic domain of human factor Xa comprising the 195-448 amino acid residues. Examples of modified active site include, but are not limited to, the catalytic domain of human factor Xa comprising 195-448 amino acid residues in SEQ ID NO: 1 with at least one amino acid substitution at position Arg306, Glu310, Arg347, Lys351, Lys414, or Arg424.

### II. Methods

It has been reported that factor Xa antidotes can reduce factor Xa inhibitor-induced bleeding in subjects, when the r-Antidote is administered at doses high enough to neutralize all plasma concentrations of the inhibitor on a continuous basis (e.g., bolus followed by infusion) and before the actual bleeding has initiated. WO/2011/008885, for instance, discloses unit dose formulations for the r-Antidote that result in an at least 1:1 molar ratio of circulating antidote over circulating factor Xa inhibitor for at least 30 minutes, administered before any actual bleeding has started.

It was believed, accordingly, that complete neutralization of the inhibitors is required because any non-neutralized inhibitor may continue or restart the bleeding. Further, continued infusion of the antidotes was thought to be necessary because the rebound of the inhibitor concentration in the plasma, in particular after the plasma concentration of the antidotes has decreased due to, for instance, natural clearance, may reinitiate bleeding. Moreover, it was unknown as to how effective the anti-bleeding activity of the r-Antidote would be once the bleeding has actually started.

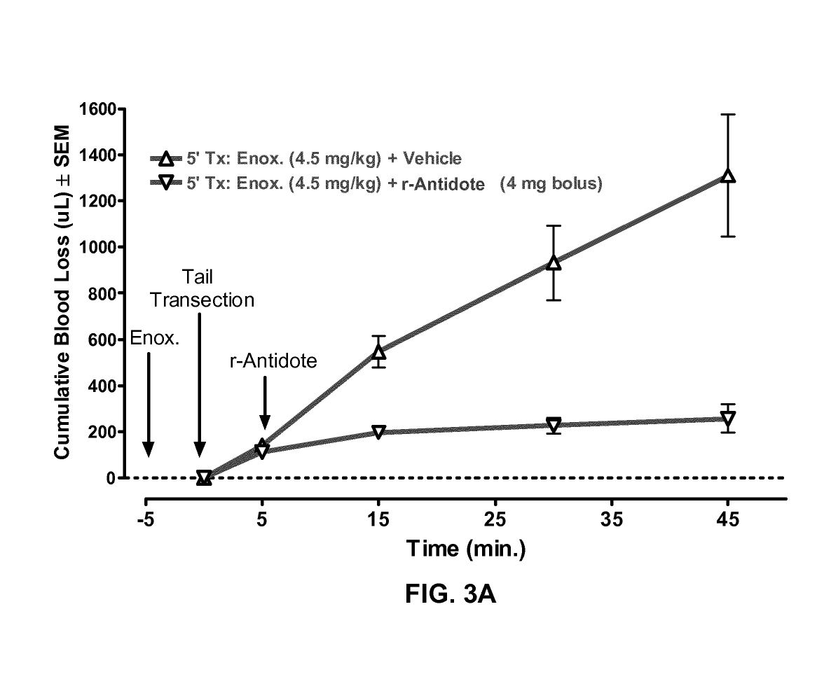

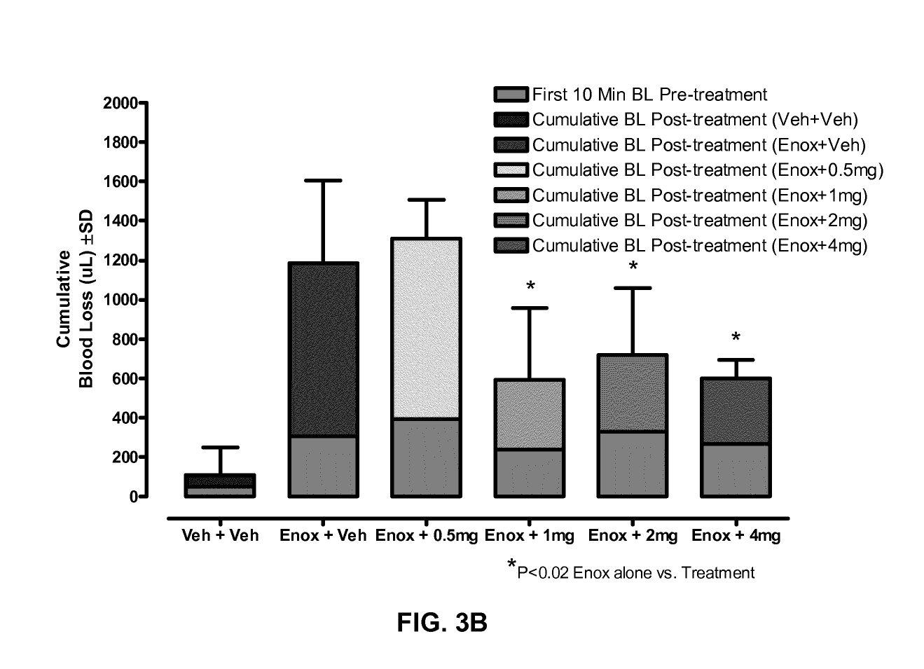

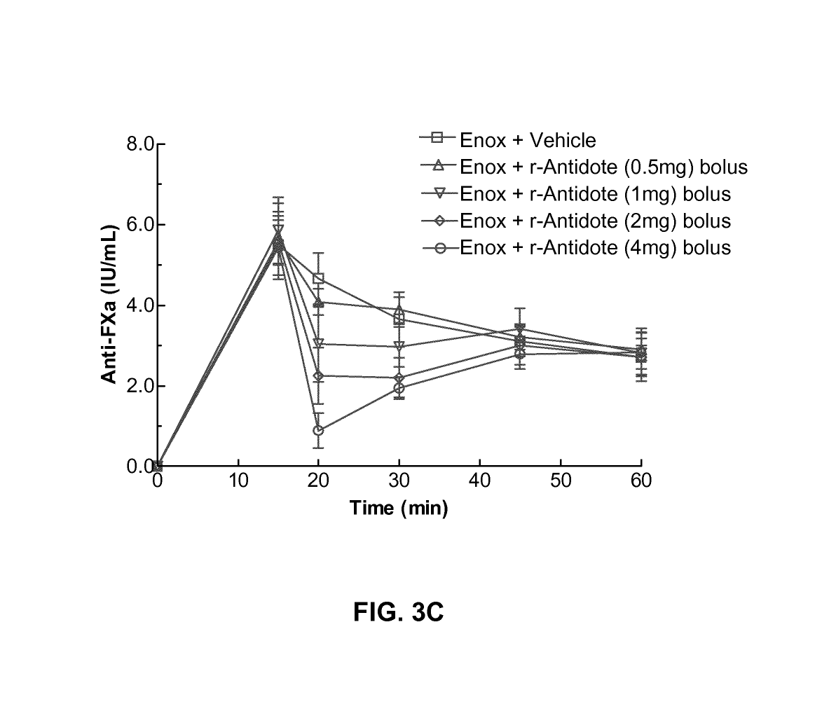

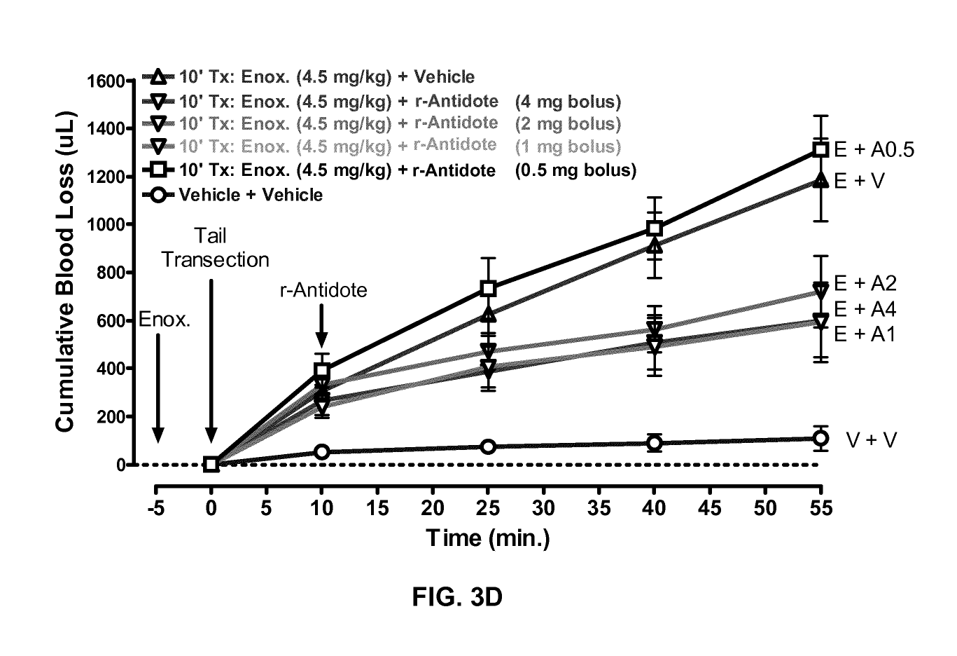

It is discovered herein, unexpectedly, that the dose of the antidotes required for effectively reversing the anticoagulant effects of factor Xa inhibitors can be lower than anticipated and lower than what was required to completely neutralize the inhibitors (see, e.g., Example 3 and FIG. 3B-D). In this respect, it is found that even partial neutralization of a factor Xa inhibitor (e.g., as low as 35% neutralization, as demonstrated in Example 3 and Table 5) can achieve a clinically effective reduction or stopping of bleeding, when a patient is in an anticoagulant therapy with an indirect factor Xa inhibitor.

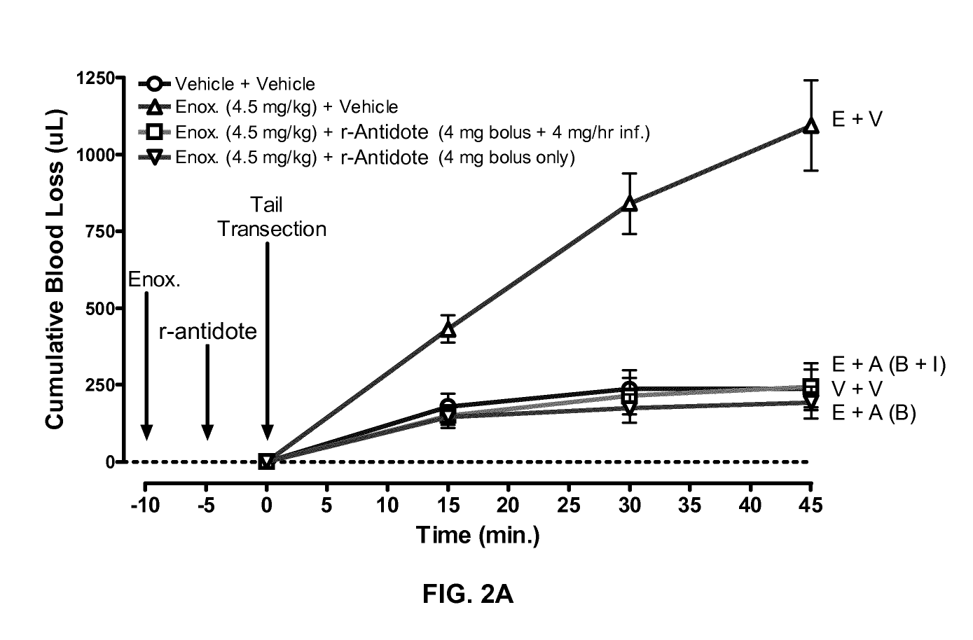

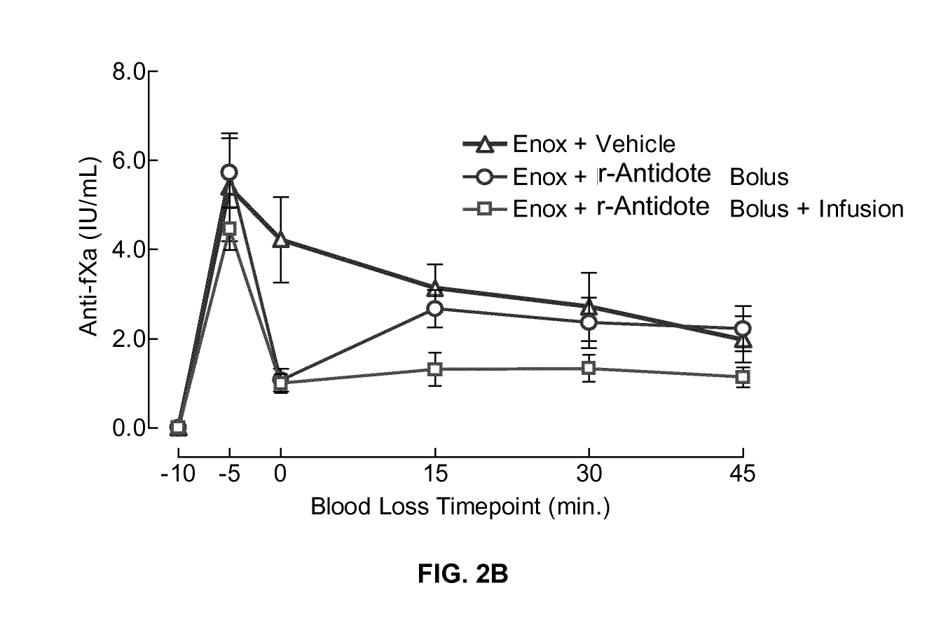

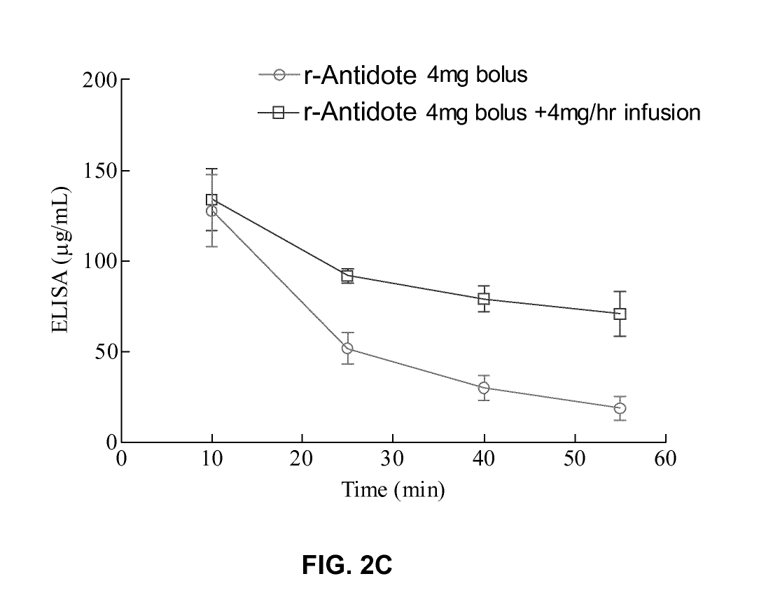

The second unexpected finding is that continued administration of factor Xa antidotes is not required for effective reduction or stopping of factor Xa inhibitor-induced bleeding (see, for instance, Examples 1-3). In other words, a transient administration of the antidote (e.g., by a single or repeated bolus only administration) provides an effective way to reduce or stop bleeding. This is surprising because, as Examples 2 and 3 show, the pharmacodynamic marker, anti-fXa activity, did rebound minutes following the administration of the antidote (FIGS. 2B and 3C). Nevertheless, bleeding did not resume (FIGS. 2A and 3B). In this context, it is noted that the half life of the r-Antidote in rats is about 15 minutes (FIG. 2C) whereas the bleeding did not resume even after 45 minutes.

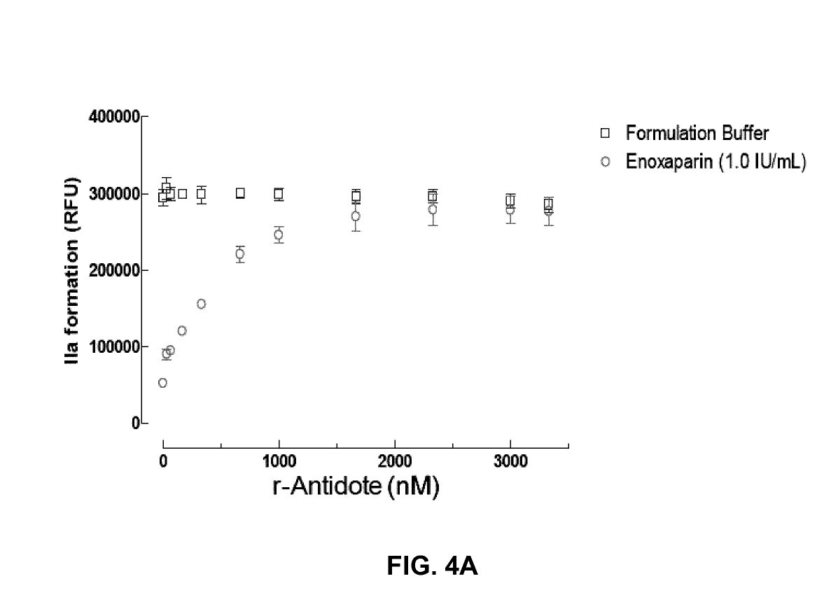

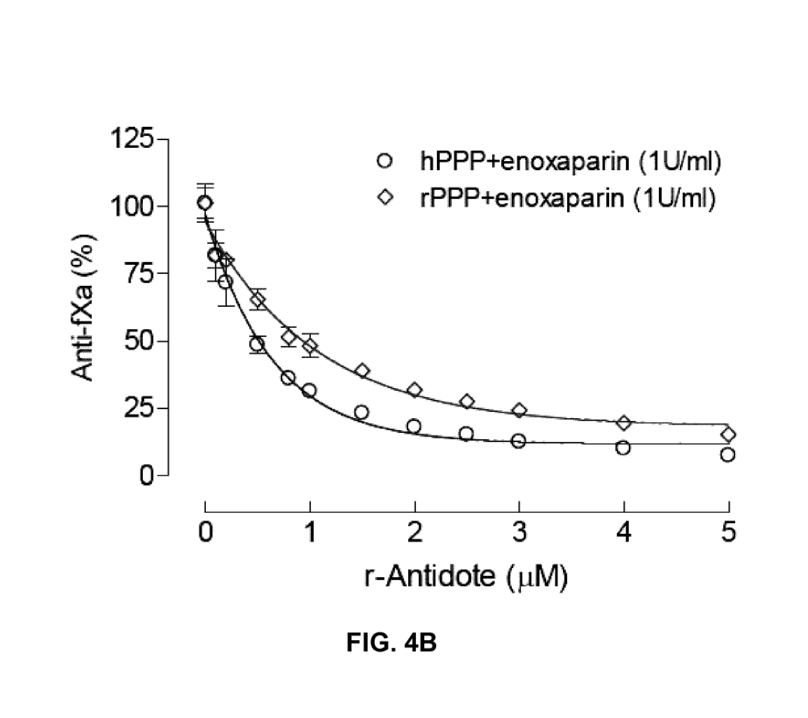

Yet another unexpected finding of the present disclosure is how effective the factor Xa antidotes can still be in reducing or stopping bleeding even when the antidotes are administered after the actual bleeding has initiated (see Example 3 and FIGS. 3A and 4). It was previously thought that an extended time of complete reversal of the anticoagulant activity is required to effectively reduce bleeding, once the bleeding is already occurring.

Based on these experimental data, it is contemplated that once anticoagulation is reversed, by a partial and transient neutralization of the antidote, normal hemostatic mechanisms are restored, a stable clot is formed and blood loss stops. Therefore, blood loss does not restart even when anticoagulation is restored. Such a mechanism is applicable to both direct and indirect factor Xa inhibitors, as shown in Examples 1-12.

Unlike direct factor Xa inhibitors that directly bind factor Xa to inhibit the activity of factor Xa, indirect factor Xa inhibitors carry out the inhibition through an inhibitor/ATIII complex. The presently disclosed experimental data suggests that, after an indirect factor Xa inhibitor, such as enoxaparin, binds to ATIII, the resulting non-covalent inhibitor/ATIII complex binds factor Xa. By virtue of such binding, factor Xa forms a covalent complex, a serpin protease complex, with ATIII. It is contemplated that this covalent complex is eliminated in the liver, through a process mediated by the serpin receptor. During the elimination, the indirect factor Xa inhibitor is freed and recycles to bind a new ATIII molecule, thereby starting another anticoagulation reaction. Therefore, a single indirect factor Xa inhibitor molecule can contribute to the inhibition of multiple factor X molecules.

Also based on the current findings, it is contemplated that the factor Xa antidotes of the present disclosure are catalytically inactive and not capable of forming a covalent complex with ATIII. Accordingly, when an antidote molecule joins an inhibitor/ATIII/antidote complex, in lieu of a factor Xa molecule, the complex is not eliminated through the liver. Consequently, the inhibitor cannot be freed from the complex and thus cannot start a new anticoagulation reaction. Therefore, by neutralizing a single molecule of an indirect factor Xa inhibitor, the antidote essentially prevents the inhibitor from carrying out multiple anticoagulant reactions. Further, the formation of the inhibitor/ATIII/antidote complex may effectively extend the half life of the antidote. This leads to sustained reversal of anticoagulation of indirect factor Xa inhibitors by the antidote.

Such an anticoagulation reversal mechanism, therefore, has not been elucidated or understood before, as it is different from, e.g., that of protamine to reverse the anticoagulation by heparins or the procoagulant strategies employed by both PCC (prothrombin complex concentrate) and factor VIIa. The protamine reversal involves charge neutralization of sulfate moieties. For PCC and fVIIa, continued administration of the agents is required to maintain circulating clotting factors to sustain hemostasis (Bershad and Suarez Neurocrit Care (2010) 12:403-413). This is particularly true when the patient has a background of anticoagulation.

Accordingly, one embodiment of the present disclosure provides a method of reducing or ceasing bleeding in a subject undergoing an anticoagulant therapy with an indirect factor Xa inhibitor. The method entails administering to the subject an amount of an antidote such that the antidote neutralizes from about 20% to about 95% of the anticoagulant activity of the inhibitor. Alternatively, in one aspect, the antidote neutralizes less than about 90%, or about 85%, 80%, 75%, 70%, 65%, 60%, 55%, 50%, 45%, 40%, 35%, 30%, or 25% of the anticoagulant activity of the inhibitor. In another aspect, the antidote neutralizes greater than about 25%, or about 30%, 35%, 40%, 45%, 50%, 55%, 60%, 65%, 70%, 75%, 80%, or 85% of the anticoagulant activity of the inhibitor. In a particular aspect, the antidote neutralizes greater than about 35% of the anticoagulant activity of the inhibitor. In some aspects, the anticoagulant activity of the inhibitor is measured as the level of an anti-fXa pharmacodynamic (PD) marker. As provided, non-limiting examples of PD markers include INR, PT, aPTT, ACT, anti fXa units, thrombin generation (Technothrombin TGA, thromboelastography, and CAT (calibrated automated thrombogram)).

Another embodiment of the present disclosure provides a method of reducing or ceasing bleeding in a subject undergoing an anticoagulant therapy with a direct factor Xa inhibitor. The method entails administering to the subject an amount of an antidote such that the antidote reaches a circulating molar concentration in the subject that is less than about 95% of the circulating molar concentration of the direct factor Xa inhibitor. Alternatively, in one aspect, the antidote reaches a circulating molar concentration in the subject that is less than about 90%, or about 85%, 80%, 75%, 70%, 65%, 60%, 55%, 50%, 45%, 40%, 35%, 30%, or 25% of the circulating molar concentration of the direct factor Xa inhibitor. In another aspect, the antidote reaches a circulating molar concentration in the subject that is greater than 25%, or about 30%, 35%, 40%, 45%, 50%, 55%, 60%, 65%, 70%, 75%, 80%, or 85% of the circulating molar concentration of the direct factor Xa inhibitor. Likewise, also provided, in one aspect, the antidote neutralizes less than about 95%, or about 90%, 85%, 80%, 75%, 70%, 65%, 60%, 55%, 50%, 45%, 40%, 35%, 30%, or 25% of the anticoagulant activity of the inhibitor. In another aspect, the antidote neutralizes greater than about 25%, or about 30%, 35%, 40%, 45%, 50%, 55%, 60%, 65%, 70%, 75%, 80%, or 85% of the anticoagulant activity of the inhibitor. The anticoagulant activity of the director inhibitor can also be measured with pharmacodynamic (PD) markers, as provided above.

Accordingly, another embodiment of the present disclosure provides a method of reducing or ceasing bleeding in a subject having received an anticoagulant therapy with a factor Xa inhibitor and experiencing clinically relevant bleeding. The method entails administering to the subject a therapeutically effective amount of a factor Xa antidote. In one aspect, the antidote is administered at least about 5 minutes after the bleeding (blood loss) initiated. Alternatively, the antidote is administered at least about 10 minutes, 15 minutes, 20 minutes, 25 minutes or 30 minutes after the bleeding has initiated. In another aspect, the amount of antidote administered neutralizes less than about 95%, or alternatively about 90%, 85%, 80%, 75%, 70%, 65%, 60%, 55%, 50%, 45%, 40%, 35%, 30%, or 25% of the inhibitor's anticoagulant activity. In another aspect, the antidote neutralizes greater than about 25%, or about 30%, 35%, 40%, 45%, 50%, 55%, 60%, 65%, 70%, 75%, 80%, or 85 of the anticoagulant activity of the inhibitor. In some aspects, the anticoagulant activity of the inhibitor is measured as the level of an anti-fXa pharmacodynamic (PD) marker.

In view of the present findings, the present disclosure further provides methods for controllably, yet safely, modulating a factor Xa inhibitor's anticoagulant activity in a subject. In one aspect, the methods entail measuring anticoagulant activity the factor Xa inhibitor in the plasma, and administering to the subject a therapeutically effective amount of a factor Xa antidote. In some aspects, the antidote neutralizes a desired amount of the circulating concentration of the factor Xa inhibitor. In one aspect, the amount of antidote administered neutralizes less than about 95%, or alternatively about 90%, 85%, 80%, 75%, 70%, 65%, 60%, 55%, 50%, 45%, 40%, 35%, 30%, or 25% of the inhibitor's anticoagulant activity. In another aspect, the antidote neutralizes greater than about 25%, or about 30%, 35%, 40%, 45%, 50%, 55%, 60%, 65%, 70%, 75%, 80%, or 85 of the anticoagulant activity of the inhibitor. In some aspects, the anticoagulant activity of the inhibitor is measured as the level of an anti-fXa pharmacodynamic (PD) marker.

In any of the above embodiments, a factor Xa antidote can be a factor Xa derivative. In one aspect, the factor Xa derivative comprises SEQ ID NO: 3 (r-Antidote), or a biological equivalent thereof. A biological equivalent of SEQ ID NO: 3, in one aspect, includes polypeptides having at least 80% sequence identity to SEQ ID NO. 3. In another aspect, such polypeptides have a modified active site and a deleted or modified Gla domain. In yet another aspect, such polypeptides do not compete with fXa in assembling into the prothrombinase complex and have reduced or no procoagulant activities.

In any of the above embodiments, a factor Xa inhibitor can be any one or more of the group consisting of fondaparinux, idraparinux, biotinylated idraparinux, enoxaparin, fragmin, tinzaparin (Innohep), low molecular weight heparin, NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

Non-limiting examples of direct factor Xa inhibitors include NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

Non-limiting examples of indirect factor Xa inhibitors include fondaparinux, idraparinux, biotinylated idraparinux, enoxaparin, fragmin, tinzaparin (Innohep), low molecular weight heparin and combinations thereof.

### III. Unit Dose Formulations

As provided above, partial and transient neutralization of a factor Xa inhibitor, with an antidote of the present disclosure, can be clinically effective in reducing or stopping the inhibitor-induced blood loss. As shown in Example 3, for a rat receiving 4.5 mg/kg enoxaparin, a single bolus dose of 1 mg r-Antidote was sufficient in reducing bleeding by 60%. Based on simple weight comparison, 1 mg r-Antidote dose for a rat (3 mg/kg) corresponds to about 200 mg for a 70 kg human. Further, as the normal dose of a factor Xa inhibitor in a human patient corresponds to about ⅕ of what was given to the rats in Examples 3, it is contemplated that a bolus dose of about 40 mg is sufficient in effectively reducing or stopping bleeding in a human patient undergoing a factor Xa inhibitor-based anticoagulant therapy, either before or after bleeding has started.

Accordingly, one embodiment of the present disclosure provides a unit dose formulation comprising a pharmaceutically acceptable carrier and from about 15 milligrams to about 95 milligrams of a factor Xa derivative. In one aspect, the unit dose formulation comprises from about 20 to about 90 mg of the factor Xa derivative. In some aspects, the amount of the factor Xa derivative in the unit dose formulation is at least about 15 mg, or 20 mg, 25 mg, 30 mg, 35 mg, 40 mg, 50 mg, or 60 mg. In some aspects, the amount of the factor Xa derivative in the unit dose formulation is no more than about 95 mg, or about 90 mg, 85 mg, 80 mg, 75 mg, 70 mg, 65 mg, 60 gm, 55 mg, 50 mg, 45 mg, 40 mg, or 35 mg. In one aspect, the unit dose is formulated for administration as a single bolus. In one aspect, the amount of the factor Xa derivative in the unit dose formulation is from about 30 mg to about 50 mg, or from about 35 mg to about 45 mg, or is about 40 mg.

In one aspect, the factor Xa derivative comprises SEQ ID NO: 3 (r-Antidote), or a biological equivalent thereof. A biological equivalent of SEQ ID NO: 3, in one aspect, includes polypeptides having at least 80% sequence identity to SEQ ID NO. 3. In another aspect, such polypeptides have a modified active site and a deleted or modified Gla domain. In yet another aspect, such polypeptides do not compete with fXa in assembling into the prothrombinase complex and have reduced or no procoagulant activities.

Methods of using the unit dose formulations are also provided. In one aspect, a method of selectively binding and inhibiting an exogenously administered factor Xa inhibitor in a subject undergoing anticoagulant therapy with a factor Xa inhibitor is provided. The method comprises administering to the subject an injection of a unit dose formulation of the present disclosure.

Another embodiment provides a method of preventing, reducing, or ceasing bleeding in a subject undergoing anticoagulant therapy with a factor Xa inhibitor comprising administering to the subject an injection of a unit dose formulation of the present disclosure.

Yet another embodiment provides a method for correcting fXa inhibitor-dependent pharmacodynamic or surrogate markers in a patient undergoing anticoagulant therapy with a factor Xa inhibitor comprising administering to the subject an injection of a unit dose formulation of the present disclosure. In some aspects, the pharmacodynamic or surrogate marker is selected from the group consisting of INR, PT, aPTT, ACT, anti fXa units, and thrombin generation.

In some aspects, the injection is given as a bolus. A “bolus” injection, as used herein, refers to an injection, that is administered relatively quickly as compared to an infusion. In one aspect, a bolus injection lasts less than 15 minutes. Alternatively, in some aspects, a bolus injection lasts less than 10 minutes, 9 minutes, 8 minutes, 7 minutes, 6 minutes, 5 minutes, 4 minutes, 3 minutes, 2 minutes or 1 minute.

“Pharmaceutically acceptable carriers” refers to any diluents, excipients, or carriers that may be used in the compositions of the disclosure. Pharmaceutically acceptable carriers include saline, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The formulations of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

In one embodiment, the antidote is lyophilized. Methods for lyophilizing polypeptides are well known in the art.

Whether lyophilized or in a solution, the formulation can be provided in forms convenient for transport or clinical use. In one embodiment, provided is a container comprising a unit of the unit dose formulation. In one aspect, provided is a unit dose package comprising a container that comprises a pharmaceutically acceptable carrier and from about 25 milligrams to about 95 milligrams of a two-chain polypeptide comprising the amino acid sequence of SEQ ID NO. 3. In some aspects, the amount of the polypeptide in the container is at least about 15 mg, or 20 mg, 25 mg, 30 mg, 35 mg, 40 mg, 50 mg, or 60 mg. In some aspects, the amount of the polypeptide in the container is no more than about 95 mg, or about 90 mg, 85 mg, 80 mg, 75 mg, 70 mg, 65 mg, 60 gm, 55 mg, 50 mg, 45 mg, 40 mg, or 35 mg. In one aspect, the amount of the polypeptide in the container is from about 30 mg to about 50 mg, or from about 35 mg to about 45 mg, or is about 40 mg.

Containers suitable for use with the present disclosure include those conventionally used in clinics. Non-limiting examples include vials, ampoules, bottles, or syringes.

Pharmaceutical formulations may also be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The formulations are for administration to a mammal, preferably a human being. Such formulations of the disclosure may be administered in a variety of ways, preferably parenterally.

It is contemplated that in order to quickly reverse the anticoagulant activity of a fXa inhibitor present in a patient's plasma in a emergency situation, the antidote of this disclosure can or may be administered to the systemic circulation via parental administration. The term “parenteral” as used herein includes subcutaneous, intravenous, intramuscular, intra-articular, intra-synovial, intrasternal, intrathecal, intrahepatic, intralesional and intracranial injection or infusion techniques. However, in cases where the fXa inhibitor being neutralized has a long plasma half life, a continuous infusion or a sustained release formulation may be required to bind to the fXa inhibitor and such free up the active fXa prior to the clearance of the fXa inhibitor from the body. Therefore, in one aspect, the formulation is administered to the subject as a bolus. In another aspect, the formulation is administered by infusion. In another aspect, the formulation is administered by a combination of bolus and infusion.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular patient will depend upon a variety of factors, including the activity of the specific antidote employed, the age, body weight, general health, sex and diet, renal and hepatic function of the patient, and the time of administration, rate of excretion, drug combination, judgment of the treating physician or veterinarian and severity of the particular disease being treated.

Polypeptides comprising the amino acid sequences of the disclosure can be prepared by expressing polynucleotides encoding the polypeptide sequences of this disclosure in an appropriate host cell. This can be accomplished by methods of recombinant DNA technology known to those skilled in the art. Accordingly, this disclosure also provides methods for recombinantly producing the polypeptides of this disclosure in a eukaryotic or prokaryotic host cells. The proteins and polypeptides of this disclosure also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the disclosure as retro-inverso peptides. In addition, the present disclosure envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁-CH₂NH-R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present disclosure provides a method to produce a constrained peptide that incorporates random sequences at all other positions.

The following non-classical amino acids may be incorporated in the peptides of the disclosure in order to introduce particular conformational motifs: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai and Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are known to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington DC; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach and Hasegawa (1996) J. Mol. Evol. 42:459-468; the block substitution matrices (BLOSUM) as described by Henikoff and Henikoff (1992) Proc. Natl. Acad. Sci. USA 89:10915-10919; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

Unless otherwise stated all temperatures are in degrees Celsius. Also, in these examples and elsewhere, abbreviations have the following meanings:

aPTT=activated partial thromboplastin time

ACT=activated clotting time

CAT=calibrated automated thrombogram

hr=hour

INR=international normalized ratio

IV=intravenous

kg=kilogram

M=molar

mg=milligram

mg/kg=milligram/kilogram

mg/mL=milligram/milliliter

min=minute

mL=milliliter

PCC=prothrombin complex concentrate

PPP=platelet poor plasma

PRP=platelet rich plasma

PT=prothrombin time

U/mL=units/milliliter

μL or uL=microliter

μM=micromolar

### Example 1

### Bolus Only Administration of r-Antidote Reverses Direct Factor Xa Inhibitor-Mediated Anticoagulation

This example shows that a single bolus dose of r-Antidote was effective in reducing blood loss by more than 80% in animals where blood loss was induced by administration of aspirin and a direct factor Xa inhibitor, rivaroxaban (Riva).

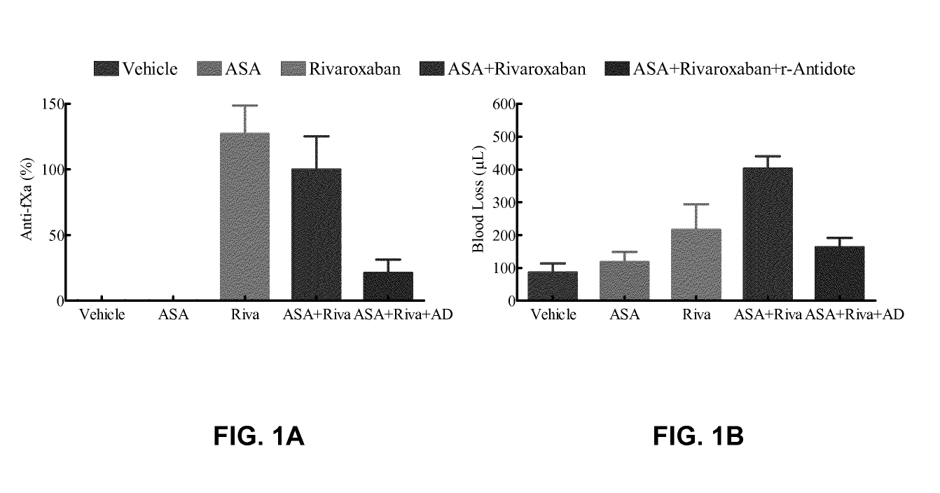

r-Antidote was tested in a mouse tail transection blood loss model for restoration of hemostasis. In aspirin (ASA)-treated mice (˜100 mg/kg/day for 5 days), single oral administration of Riva (Riva+ASA, 50 mg/Kg Riva) increased blood loss 3.4-fold (Riva+ASA vs. ASA, p=0.0003). Intravenous administration of r-Antidote (AD) (Riva+ASA+AD, 0.96mg AD/mouse) two hours after the oral Riva dose reduced blood loss by ˜84% (Riva+ASA+AD vs. Riva+ASA, p=0.0002) (FIG. 1B). Decreased blood loss correlated with r-Antidote plasma concentrations, reduction of the pharmacodynamic (PD) marker, anti-fXa activity (FIG. 1A), and whole blood International Normalized Ratio (INR).

These results show that a single bolus administration of r-Antidote can be as effective as bolus plus infusion administrations of the r-Antidote to reduce or stop a direct factor Xa-induced bleeding, in which the bolus doses are the same.

### Example 2

### Bolus Only Administration of r-Antidote Reverses Indirect Factor Xa Inhibitor-Mediated Anticoagulation Through Transient Neutralization of the Inhibitor

This example shows that r-Antidote is also effective in reducing or stopping bleeding induced by indirect factor Xa inhibitors. Further, even a transient neutralization of the inhibitor by the r-Antidote is effective in reducing or stopping the bleeding.

In a rat blood loss model, anesthetized rats received 4.5 mg/kg intravenously (IV) administration of enoxaparin (or vehicle) followed, 5 min later, by a single bolus (prophylactic) administration of r-Antidote (4 mg bolus), a bolus administration of r-Antidote along with r-Antidote infusion (4 mg bolus +4 mg/hr infusion), or vehicle. Then, tail transection was initiated another 5 min later, and cumulative blood loss was measure for up to 45 minutes. Plasma levels of r-Antidote and pharmacodynamic (PD) marker, anti-fXa activity, were determined at multiple time points.

The data show that bolus only versus a bolus plus infusion r-Antidote administrative regimen both were equally effective with no blood loss greater than vehicle (no enoxaparin) alone (Table 4 and FIG. 2A) despite the rebound in anti-fXa activity with the bolus only regimen (FIG. 2B). Such rebound in the anti-fXa activity correlated with the decrease of the plasma concentrations of the r-Antidote in the bolus only regimen (FIG. 2C), due to the r-Antidote's relatively short half life in rats, which was observed to be about 15 minutes.

This example suggests that once anticoagulation is reversed by a transient administration (bolus only) of r-Antidote, and normal hemostatic mechanisms are restored, a stable clot is formed. At this point, the blood loss stops and subsequent restoration of anticoagulation does not reinitiate blood loss.

TABLE 4
Cumulative blood loss after tail transection

| | Cumulative Blood Loss (μL) | | |
|---|---|---|---|
| Time Point: | 0′-15′ | 0′-30′ | 0′-45′ |
| Vehicle + Vehicle | 179 | 237 | 238 |
| Enox + r-Antidote | 149 | 214 | 244 |
| (bolus + infusion) | | | |
| Enox + r-Antidote | 147 | 174 | 192 |
| (bolus only) | | | |
| Enox + Vehicle | 432* | 842* | 1095* |

*p ≤ 0.003 (vs. all remaining groups within the respective time point)

### Example 3

### Bolus Only Administration of r-Antidote Reverses Indirect Factor Xa Inhibitor-Mediated Anticoagulation Following Initiation of Bleeding Through Partial and Transient Neutralization of the Inhibitor

This example shows that a single bolus administration of r-Antidote reversed PD marker and blood loss induced by enoxaparin administration in a rat treatment model of blood loss. This example also shows that, contrary to the conventional understanding, (a) the dose of r-Antidote required for reversing the anticoagulant effects of indirect factor Xa inhibitors can be lower, (b) a follow up infusion of the antidote is not required and (c) administration of the antidote even after bleeding has initiated can still be effective in reducing or stopping the bleeding.

In a pilot study using this antidote treatment model, anesthetized rats were intravenously (IV) administered either vehicle or enoxaparin (4.5 mg/kg, IV bolus) followed by tail transection at 5 min post-dosing. After 5 minutes of blood loss, an injection of vehicle or r-Antidote (4 mg/rat) was administered. Within 5 minutes after bolus injection of r-Antidote, bleeding was significantly reduced or stopped for all rats. The cumulative blood loss in the r-Antidote group was less than 20% of that in the enoxaparin only group, at the end of the experiment (FIG. 3A).

To investigate the effects of r-Antidote under more aggressive bleeding circumstance, the rat tail transection model was modified where the tail was transected and allowed to bleed for 10 minutes prior to treatment. Administration of r-Antidote (0.5, 1, 2 or 4 mg) or vehicle was initiated at 15 min (10 min after tail injury) by IV bolus injection (n=7/group). Blood loss was measured for an additional 45 min following vehicle or r-Antidote administration. Plasma levels of r-Antidote and anti-fXa activity were determined at multiple time points.

Anticoagulation with enoxaparin (4.5 mg/kg) increased blood loss by ˜11-fold over vehicle (FIG. 3B). It is worth noting that such a dose of enoxaparin would result in a plasma concentration in a human patient that is about 5 fold of the plasma concentration in the typical clinical setting. Total blood loss in the enoxaparin-treated group represented ˜5% of the animal's total blood volume with ˜26% of blood loss occurring within the first 10 min prior to treatment.

Administration of r-Antidote at 1, 2, or 4 mg produced a significant reduction in cumulative blood loss (60, 56 and 62%, respectively; Enox+r-Antidote vs. Enox+vehicle: p≤0.04 for all 3 groups) with complete cessation of bleeding in 70-85% of animals (FIGS. 3B and 3D and Table 5). The 0.5 mg dose of r-Antidote, in contrast, did not reduce bleeding (FIGS. 3B and 3D and Table 5).

As shown in FIG. 3C and Table 5, the PD marker, anti-fXa activity, was dose-dependently reversed by 13, 35, 52 and 81% at 5 min following administration of r-Antidote at 0.5, 1, 2 and 4 mg, respectively. As measured, such doses of the r-Antidote resulted in r-Antidote peak plasma concentrations of 0.45, 1.1, 1.8 and 4.3 μM, respectively (Table 5).

These data demonstrate that even a 35% neutralization of the anticoagulant activity of the indirect factor Xa inhibitor, as measured with the PD markers, was effective to reduce the indirect factor Xa inhibitor-induced bleeding.

TABLE 5
Peak plasma concentrations of factor Xa
inhibitor, ATIII complex and r-Antidote

| | | | Decrease | Reduction |
|---|---|---|---|---|
| Experimental | | Peak Plasma | in anti-fXa | of blood |
| Component | Dose | Concentration | activity | loss |
| Enoxaparin | 4.5 mg/kg | 10-14 μM | N/A | N/A |
| | | (estimated) | | |
| ATIII | — | 2-4 μM | N/A | N/A |
| (endogenous rat) | | (estimated) | | |
| r-Antidote - | 4 mg | 4.3 μM | 81% | 62% |
| high dose | | | | |
| r-Antidote - | 2 mg | 1.8 μM | 52% | 56% |
| mid dose | | | | |
| r-Antidote - | 1 mg | 1.1 μM | 35% | 60% |
| low dose | | | | |
| r-Antidote - | 0.5 mg | 0.45 μM | 13% | 0% |
| very low dose | | | | |

Therefore, this example shows, surprisingly, that even a partial neutralization (˜25-50%) of anti-fXa activity by a transient, single IV bolus administration of r-Antidote following injury was sufficient to reduce or completely stop bleeding even though anti-fXa activity was gradually increased in later time points due to clearance of r-Antidote and residual anticoagulation. These data demonstrate that a single (or repeat if necessary) bolus administration of r-Antidote is able to reverse anticoagulation and impact bleeding related to indirect fXa inhibitors, such as enoxaparin. Further, the r-Antidote can still be effective in reducing or stopping bleeding even after the bleeding has initiated, making the r-Antidote suitable for emergency use.

### Example 4

### Low Does r-Antidote Reverses Blood Loss and PD Markers

This example reports additional experimental data obtained from the animal model used in Example 3.

Anesthetized rats were administered enoxaparin (4.5 mg/kg) or vehicle by I.V. bolus injection followed by tail transection (2 mm from tip) 5 minutes later. Treatment with r-Antidote (0.5, 1, 2 or 4 mg) or vehicle was given at 15 minutes (10 minutes after tail transection). The tip of the tail was placed in physiologic saline at 37° C. and blood collected for an additional 45 minutes after tail transection. Blood was lysed by diluting in water and freezing at −80° C. overnight. The hemoglobin concentration in the resulting solutions was measured by absorbance (OD490) and compared against a standard curve constructed with known volume of blood under the same condition to estimate volume of blood loss. r-Antidote plasma concentration was measured by a paired polyclonal antibodies recognizing human fX/fXa. Anti-fXa activity of enoxaparin was measured using a modified chromogenic LMW heparin kit. Tissue factor (TF)-initiated thrombin generation was carried out in human plasma using a fluorogenic thrombin substrate by measurement of change of relative fluorescence unit (RFU) for 10 min following initiation of the reaction.

The enoxaparin-induced blood loss and its reversal by r-Antidote are shown in FIG. 3B-D. Table 6 shows the average anti-fXa activity in enoxaparin anticoagulated rats and r-Antidote plasma concentration.

TABLE 6
Average anti-fXa activity and r-Antidote plasma concentration

| | Time | | r-Antidote | | | |
|---|---|---|---|---|---|---|
| | Point | Vehicle | 0.5 mg | 1 mg | 2 mg | 4 mg |
| Anti-fXa | 15 | 5.4 | 5.6 | 5.9 | 5.6 | 5.4 |
| Activity | 20 | 4.7 | 4.1 | 3.0 | 2.1 | 0.9 |
| (IU/mL) | 30 | 3.3 | 3.9 | 3.0 | 2.2 | 1.9 |
| | 45 | 3.0 | 3.2 | 3.41 | 3.0 | 2.78 |
| | 60 | 2.8 | 2.9 | 2.8 | 2.7 | 2.8 |
| r-Antidote | 20 | 0.0 | 0.5 | 1.1 | 1.8 | 4.3 |
| plasma | 30 | 0.0 | 0.3 | 0.6 | 1.2 | 2.4 |
| (μM) | 45 | 0.0 | 0.2 | 0.4 | 0.6 | 1.3 |
| | 60 | 0.0 | 0.1 | 0.2 | 0.3 | 0.7 |

Table 6, therefore, shows that the reduction percentage in anti-fXa activity corresponded to plasma concentration of r-Antidote at the end of the 5-minute bolus administration.

Further, in an in vitro experiment, r-Antidote showed reversed the enoxaparin mediated inhibition of thrombin generation in human plasma (FIG. 4A). Also, in both rat (rPPP) and human (hPPP) platelet poor plasma (PPP), r-Antidote dose-dependently reversed the anti-fXa activity of therapeutic concentration of enoxaparin (FIG. 4B).

Together, the data in Examples 3 and 4 demonstrate that a partial and transient reversal of enoxaparin-induced anticoagulation was sufficient to reduce bleeding in a clinically relevant model of haemostasis. The partial reversal of anti-fXa activity (35% in the case of the 1 mg r-Antidote bolus dose) for a short period of time was sufficient to reduce or halt bleeding. These data suggest that a single bolus administration of r-Antidote has the potential to rapidly reduce blood loss and reverse pharmacodynamic markers in patients under anticoagulation with fXa inhibitors.

### Example 5

### r-Antidote but not rfVIIa Reverses Rivaroxaban Induced Anticoagulation as Measured by Reduction in Blood Loss in a Rabbit Liver Laceration Model

This example used a modified rabbit liver laceration model to demonstrate the effects of r-Antidote (PRT), a recombinant fXa derivative, to reverse rivaroxaban induced anticoagulation as measured by reduction of blood loss, decrease of unbound fraction of rivaroxaban in plasma and relevant pharmacodynamic markers: anti-fXa activity, prothrombin (PT) and activated thromboplastin (aPTT) times. Recombinant fVIIa (rfVIIa) was tested in the same model for comparison.

Anesthetized rabbits were administered vehicle or 1 mg/kg rivaroxaban via IV bolus injection. After 30 minutes to allow the rivaroxaban to biodistribute, vehicle, r-Antidote or rfVIIa was administered as an injection followed by laceration of two liver lobes (10×1-cm long and 3-mm deep incisions with scalpel blade) and blood loss collected on pre-weighed gauze over 15 minutes. Blood loss due to rivaroxaban anticoagulation represented approximately 10% of the animal's total blood volume. Total rivaroxaban concentration in plasma was measured by LC-MS/MS. Free fraction of rivaroxaban (non-protein, non-r-Antidote bound) was first separated using ultra-filtration followed by LC-MS/MS quantitation. Anti-fXa activity was measured using a modified chromogenic LMW heparin kit. PT and aPTT was measured using commercial reagents (HemoslL).

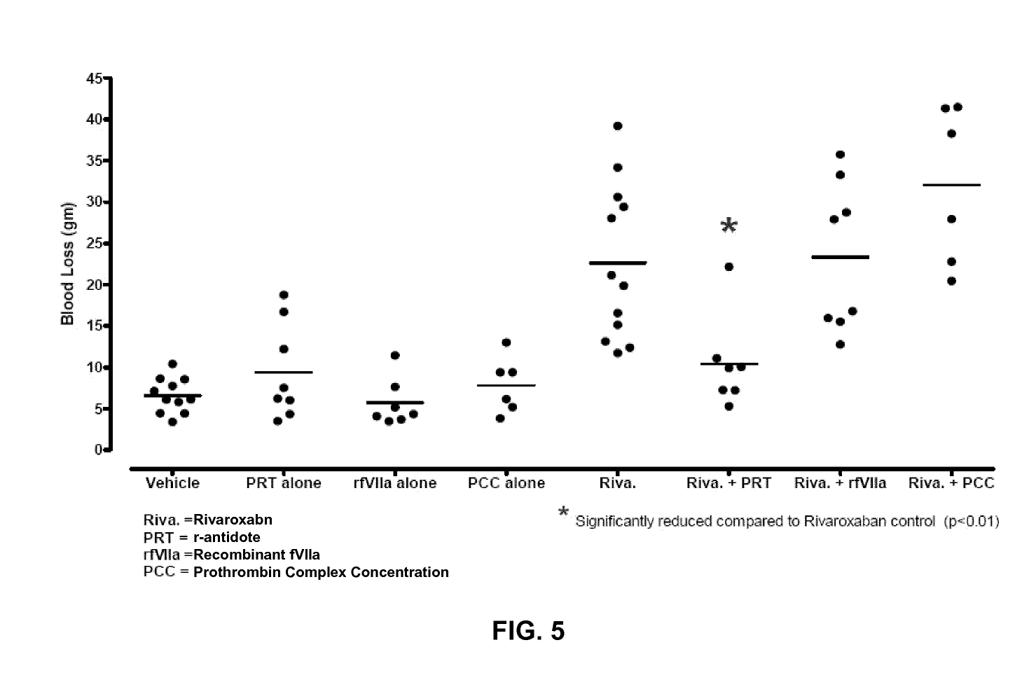

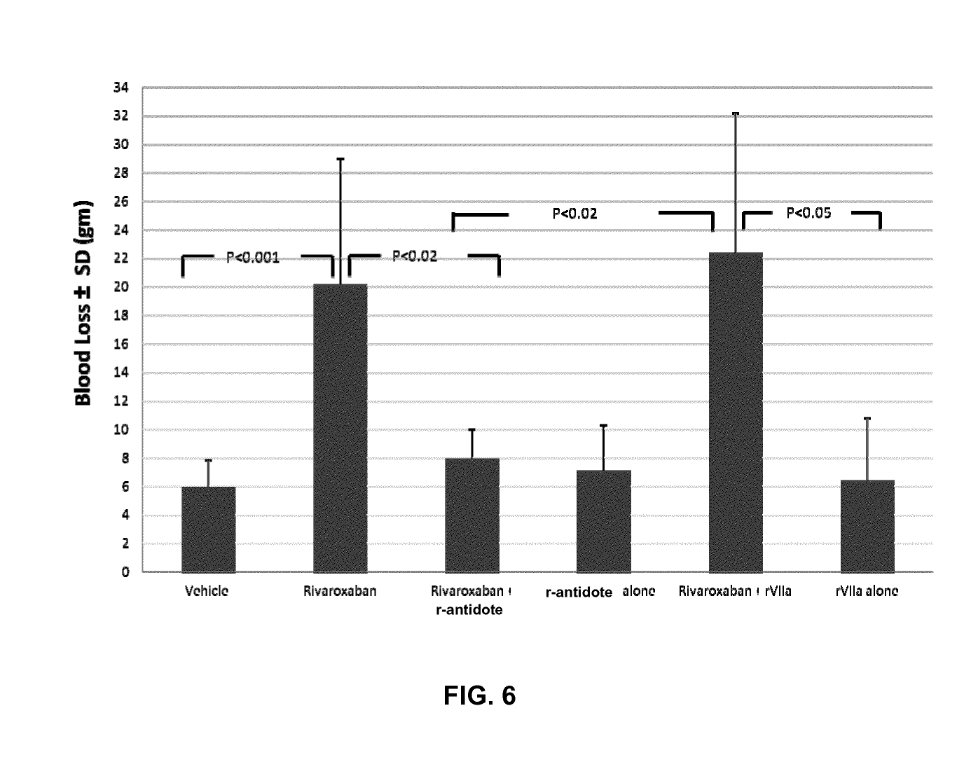

Anticoagulation by rivaroxaban (1.65 μM average plasma concentration at 30 min time point) resulted in 2.3-fold and 1.9-fold prolongation of PT and aPTT, respectively and increased blood loss by 3.2-fold over vehicle (FIGS. 5 and 6). r-Antidote (75 mg/rabbit) administration reduced blood loss by >85% and decreased peak anti-fXa activity by 98%, PT by 74%, aPTT by 66% and the free fraction of rivaroxaban in plasma from 26 to <0.5%. In contrast, rfVIIa (150 μg/kg) had no effect on blood loss but reversed PT by 86% and aPTT by 56%. r-Antidote and rfVIIa alone had no effect on blood loss, but rfVIIa decreased PT by 34%.

Tables 7 and 8 show that rivaroxaban anticoagulation resulted in 2.5-fold prolongation of PT (Table 7) and 1.8-fold prolongation of aPTT (Table 8) at the 30 minute time point. Administration of r-Antidote and rfVIIa reduced the PT prolongation by 81% and 84% at the 35 minute time point, respectively. Administration of rfVIIa alone decreased mean PT values by 32%. Administration of r-Antidote and rfVIIa reduced the aPTT prolongation by 60% and 33%, respectively. The reduction in PT and aPTT with administration of rfVIIa did not correlate with a reduction in blood loss. Administration of PCC (prothrombin complex concentrate) had no effect on PT but showed a delayed reduction aPTT by 36% at the 50 minute time point.

TABLES 7
Measured prothrombin times
Prothrombin Times (seconds)

| | Time point | | | |
|---|---|---|---|---|
| Treatment | (minutes post rivaroxaban) | | | |
| Regimen | 0 | 30 | 35 | 50 |
| Vehicle + Vehicle | 6.5 ± 0.5 | 6.6 ± 0.3 | 6.9 ± 0.4 | 6.9 ± 0.4 |
| Vehicle + r-Anti- | 6.4 ± 0.3 | 6.3 ± 0.3 | 6.6 ± 0.3 | 6.5 ± 0.3 |
| dote | | | | |
| Vehicle + rfVIIa | 6.3 ± 0.4 | 6.5 ± 0.5 | 4.4 ± 0.2 | 4.4 ± 0.3 |
| Vehicle + PCC | 7.1 ± 0.4 | 6.9 ± 0.3 | 7.8 ± 0.4 | 7.9 ± 0.4 |
| Rivaroxaban + | 6.2 ± 0.4 | 15.8 ± 1.5 | 15.0 ± 1.6 | 13.8 ± 1.3 |
| Vehicle | | | | |
| Rivaroxaban + | 6.7 ± 0.6 | 17.1 ± 2.1 | 8.7 ± 0.9 | 12.5 ± 1.1 |
| r-Antidote | | | | |
| Rivaroxaban + | 6.3 ± 0.5 | 15.6 ± 1.4 | 7.8 ± 1.0 | 7.4 ± 0.8 |
| rfVIIa | | | | |
| Rivaroxaban + | 6.7 ± 0.2 | 17.4 ± 1.6 | 16.8 ± 1.6 | 15.5 ± 1.1 |
| PCC | | | | |

TABLE 8
Measured activated partial thromboplastin times
Activated Partial Thromboplastin Times (seconds)

| | |
|---|---|
| | Time point |
| Treatment | (minutes post rivaroxaban) |

| Regimen | 0 | 30 | 35 | 50 |
|---|---|---|---|---|
| Vehicle + Vehicle | 26 ± 4 | 27 ± 3 | 27 ± 3 | 26 ± 3 |
| Vehicle + r-Anti- | 28 ± 4 | 27 ± 4 | 27 ± 3 | 25 ± 3 |
| dote | | | | |
| Vehicle + rfVIIa | 26 ± 2 | 27 ± 2 | 25 ± 3 | 24 ± 3 |
| Vehicle + PCC | 23 ± 5 | 25 ± 5 | 29 ± 6 | 24 ± 6 |
| Rivaroxaban + | 28 ± 5 | 49 ± 8 | 48 ± 8 | 48 ± 8 |
| Vehicle | | | | |
| Rivaroxaban + | 27 ± 6 | 49 ± 8 | 36 ± 5 | 40 ± 6 |
| r-Antidote | | | | |
| Rivaroxaban + | 23 ± 3 | 43 ± 5 | 39 ± 4 | 35 ± 4 |
| rfVIIa | | | | |
| Rivaroxaban + | 24 ± 4 | 43 ± 6 | 46 ± 4 | 36 ± 11 |
| PCC | | | | |

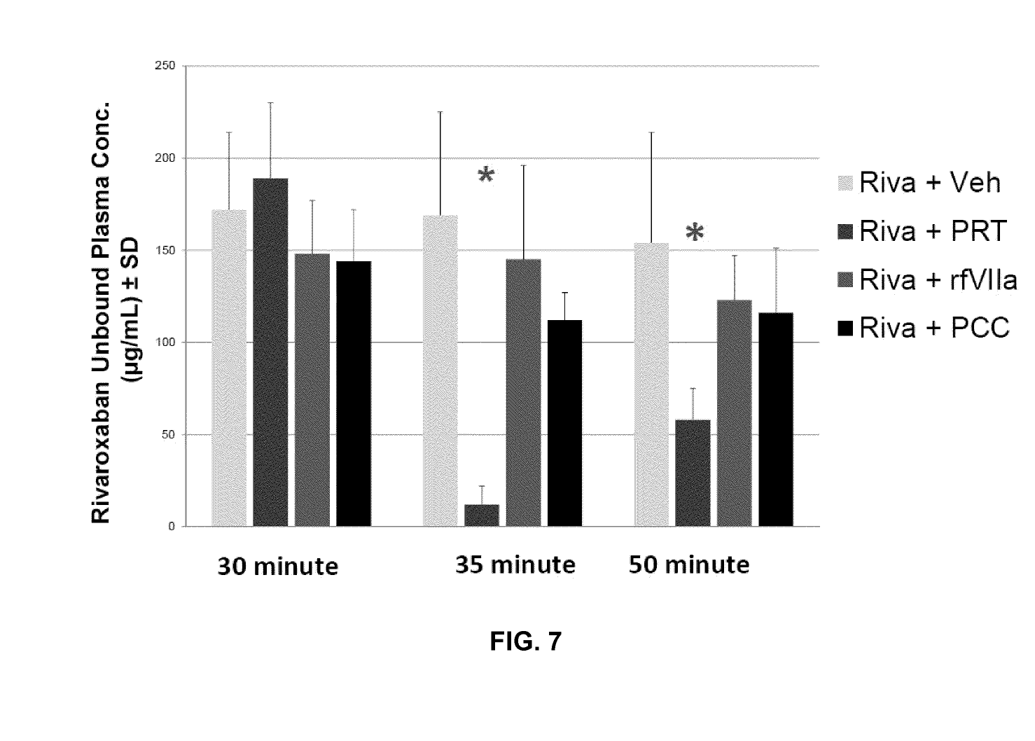

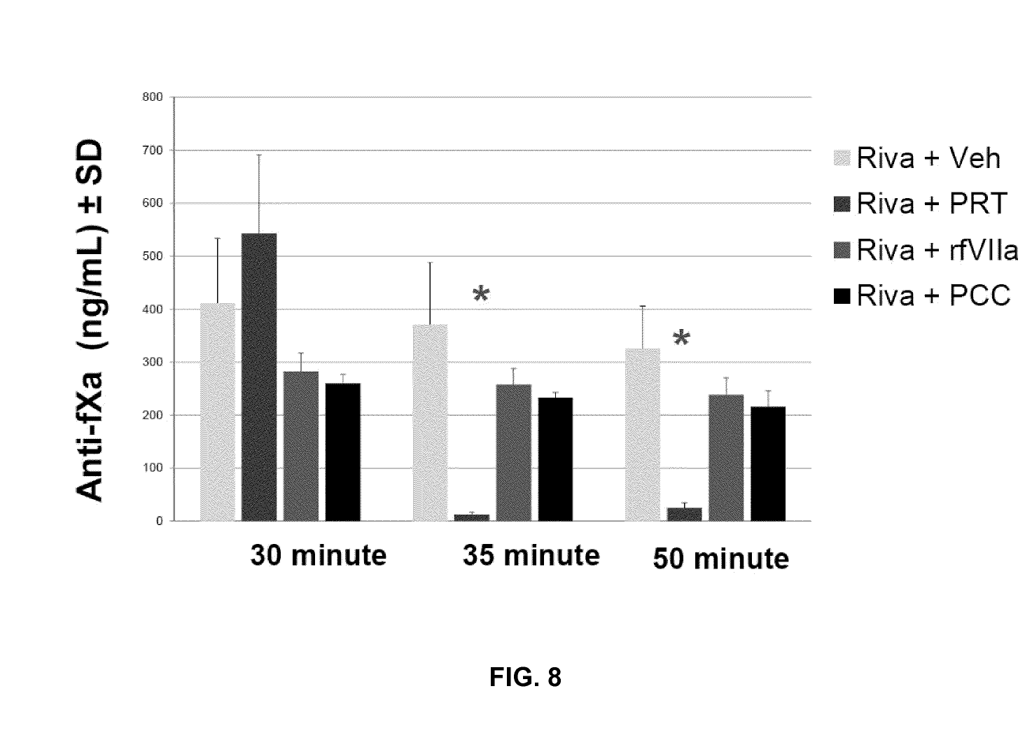

These data demonstrate that r-Antidote can reduce blood loss due to rivaroxaban induced anticoagulation using a single bolus administration. Reduction of blood loss with r-Antidote correlated to the decrease in the free fraction of rivaroxaban in plasma (FIG. 7) as well as PD markers anti-fXa activity and PT (FIG. 8), while rfVIIa decreased PT but had no effect on blood loss.

### Example 6

### Reversal of Direct fXa Inhibitors by r-Antidote

In this example, the potency (Kd) of r-Antidote binding to three inhibitors was tested and compared to the inhibition constants (Ki) reported for the inhibitors against human plasma derived fXa.

To measure the inhibition of fXa activity by direct fXa inhibitors and reversal of its inhibitory effect by r-Antidote, purified human plasma fXa (3 nM) (available commercially from Haematologic Technologies), varying concentrations of inhibitor (0, 2.5, 5.0 and 7.5 nM) and r-Antidote were added to assay buffer (20 mM Tris, 150 mM NaCl, 5 mM Ca²⁺, 0.1% BSA, pH=7.4). After incubation at room temperature (RT) for 30 min, 100 μM Spectrozyme-fXa (available commercially from American Diagnostica) was added to the mixture and initial rate of substrate cleavage was monitored continuously for 5 min at 405 nm in a 96-well plate reader. Initial velocity of product formation as a function of inhibitor and r-Antidote concentration was analyzed by Dynafit (available commercially from Biokin) to estimate the binding affinity of r-Antidote to each inhibitor. Betrixaban was synthesized according to methods known in the art. Rivaroxaban and apixaban can be purchased commercially from, for example, J Star Research and American Custom Chemicals Corporation.

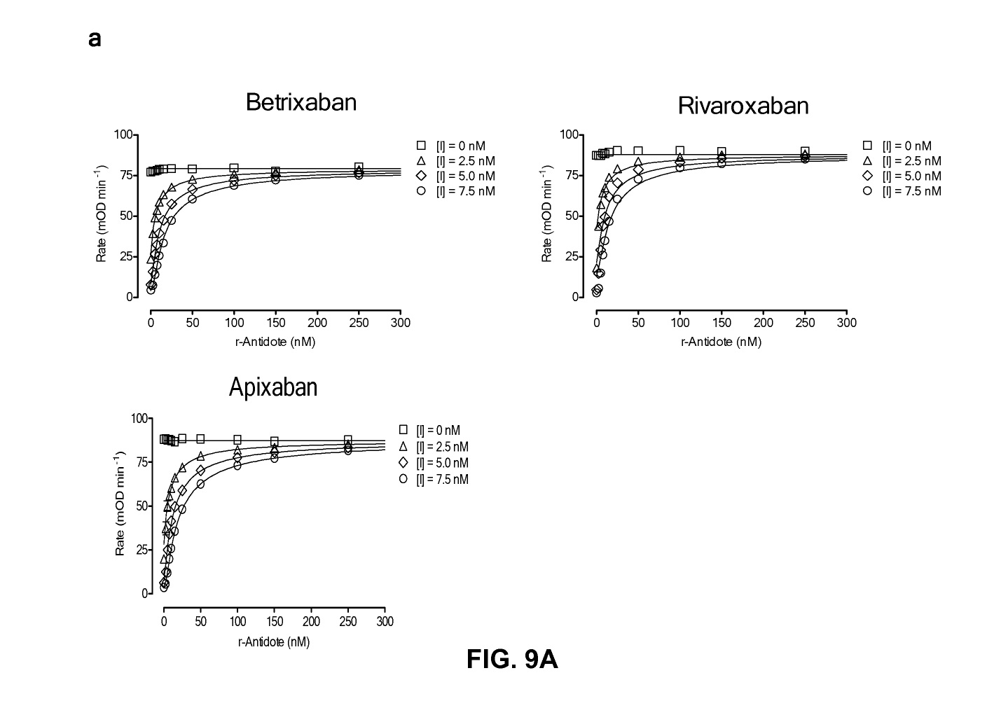

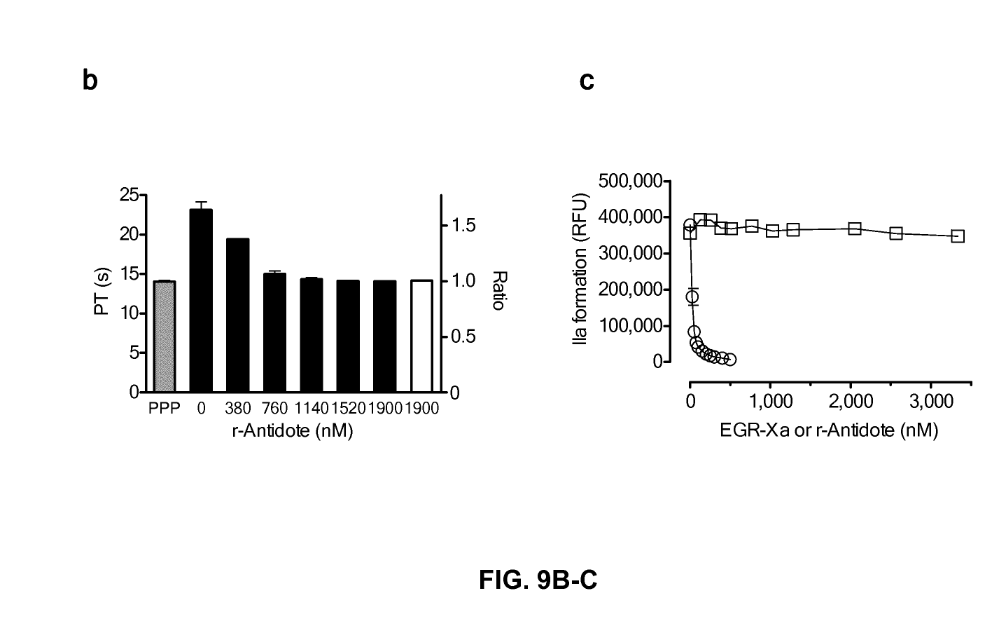

The three panels of FIG. 9A show reversal of betrixaban, rivaroxaban and apixaban inhibition using purified human plasma derived fXa and peptidyl substrate. While r-Antidote dose-dependently reversed the inhibitory activity of small molecule fXa inhibitors, it did not produce any change in fXa activity in the absence of inhibitor. This observation is consistent with r-Antidote's expected lack of catalytic activity. Analysis of these kinetic data indicated that r-Antidote has sub-nanomolar affinities for the direct fXa inhibitors. The relative potency of binding is in the same order of magnitude as the inhibition constants reported previously in the art.

Following testing in a purified enzyme system, the ability of r-Antidote to reverse the effects of anticoagulation in plasma was evaluated. Residual inhibitory activity of rivaroxaban, as measured by anti-fXa activity, was determined after incubation with different amount of r-Antidote and normalized to baseline values. Inhibitory activity was dose-dependently and completely reversed by r-Antidote in both human and rat plasma (data not shown). Rivaroxaban was then used as a prototype inhibitor to test the effects of r-Antidote on markers of anticoagulation as measured by ex vivo clotting assays. FIG. 9B shows that r-Antidote reversed the anticoagulant effects of rivaroxaban in human plasma. At a near peak therapeutic concentration, rivaroxaban (1 μM) caused a prolongation of prothrombin time (PT). This prolongation was corrected to baseline levels by an approximately equal molar concentration of r-Antidote. While r-Antidote bound and neutralized the anticoagulant effect of rivaroxaban in a dose-dependent manner, it alone did not change clotting time at the highest concentration tested. Thus, r-Antidote alone did not produce detectable levels of procoagulant or anticoagulant activity as measured by this clotting assay. As an additional measure of pro or anticoagulant activity, a thrombin generation assay which tests fXa activity in the presence of cofactor factor Va and phospholipids in the prothrombinase complex was used. r-Antidote lacks the GLA-domain and should have substantially reduced capacity to incorporate into the prothrombinase complex. In contrast, active-site inhibited fXa with an intact GLA-domain is expected to be a potent anticoagulant, as it can compete with endogenous fXa for assembly into the prothrombinase complex. FIG. 9C compares the effect of r-Antidote with EGR-Xa, an active site inhibited full length human fXa, on prothrombinase activity. Prothrombinase activity was measured by thrombin generation in human plasma with increasing concentrations of r-Antidote (0-3.3 μM) or EGR-Xa (0-0.5 μM). Formation of thrombin (relative fluorescence units, RFU) in the presence of r-Antidote remained essentially unchanged up to a concentration of 3.3 μM, while EGR-Xa showed potent inhibition in the same assay (IC₅₀=26 nM). These results are consistent with earlier reports of active site-inhibited native fXa for the inhibition of prothrombinase activity.

For the thrombin generation assay in human plasma, pooled human plasma in 0.32% citrate (75 μL) was mixed with CaCl₂ and Z-Gly-Gly-Arg-aminomethylcoumarin (Z-GGR-AMC, available commercially from Bachem) fluorogenic thrombin substrate. Innovin was used as the source of TF to initiate the generation of thrombin. The reaction mixture (final volume=100 μL) contained 15 mM Ca²⁺, 100 μM Z-GGR-AMC, and 0.1 nM TF. Thrombin formation was monitored continuously at 37° C. in a 96-well fluorescence plate reader measuring the relative fluorescence units (RFU). r-Antidote and EGR-Xa, an active site-inhibited human plasma derived fXa (available commercially from Haematologic Technologies), when present, were pre-incubated with plasma for 20 min at RT before initiation of thrombin generation. Tris-buffered saline (20 mM Tris, 150 mM NaCl, pH=7.4) was used to adjust the final volume of the reaction mixture.

For human in vitro and rat ex vivo experiments, PT was measured using MLA Electra 800 automatic coagulation timer. Innovin was automatically dispensed to plasma samples (100 μL) per manufacturer's instructions. FXa inhibitor and r-Antidote, when present, was pre-incubated with human plasma at room temperature for 20 min before initiation of the clotting measurements. For rabbit experiments, PT and aPTT were measured using a Beckman Coulter ACL Elite instrument with HemoslL reagents (available commercially from Instrumentation Laboratories). Whole blood INR (International Normalized Ratio) for animal samples were measured by Hemchron Jr. Signature Cartridges (International Technidyne Corporation) according to manufacturer's instructions.

### Example 7

### r-Antidote Reverses the Pharmacodynamic Effect of fXa Inhibitor in Vivo

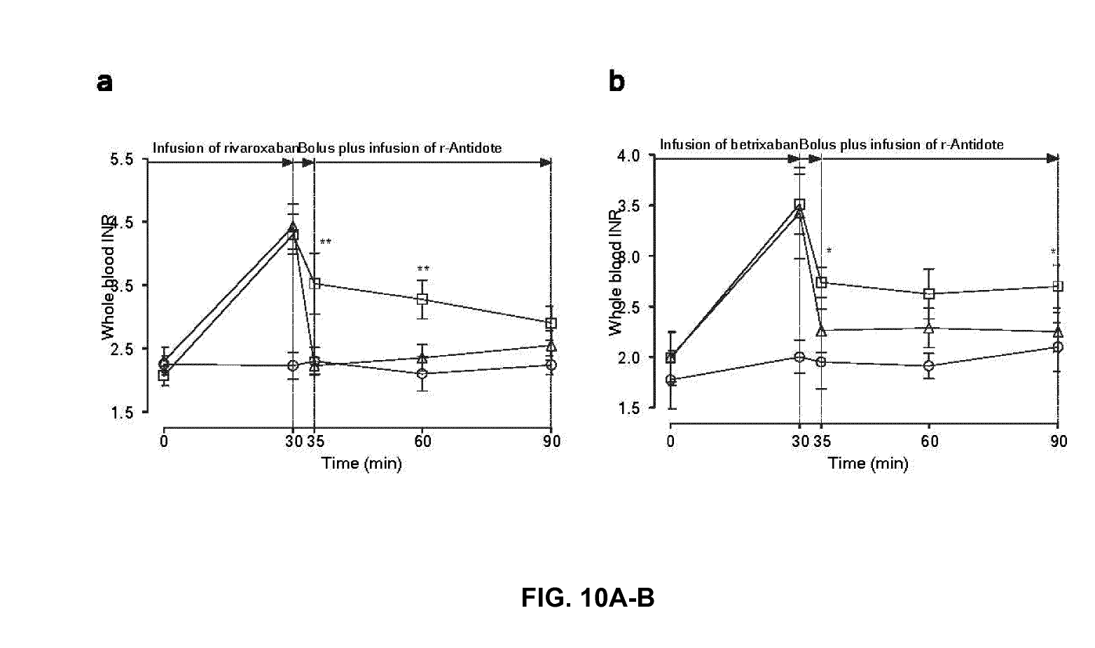

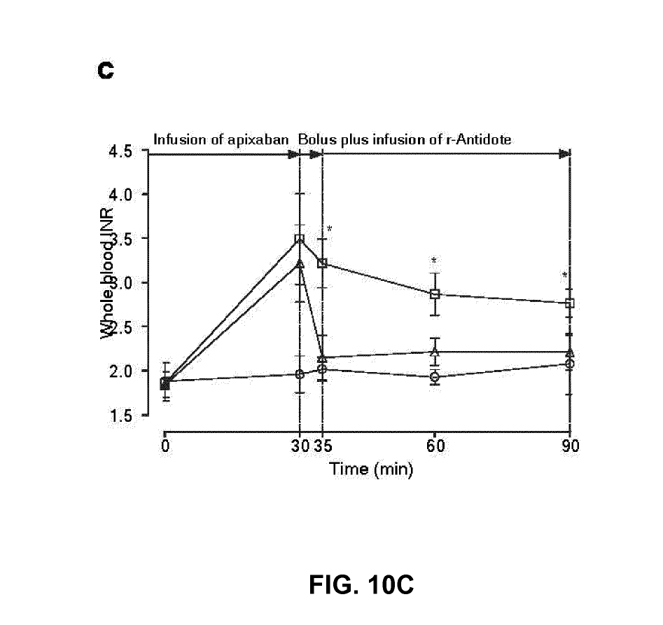

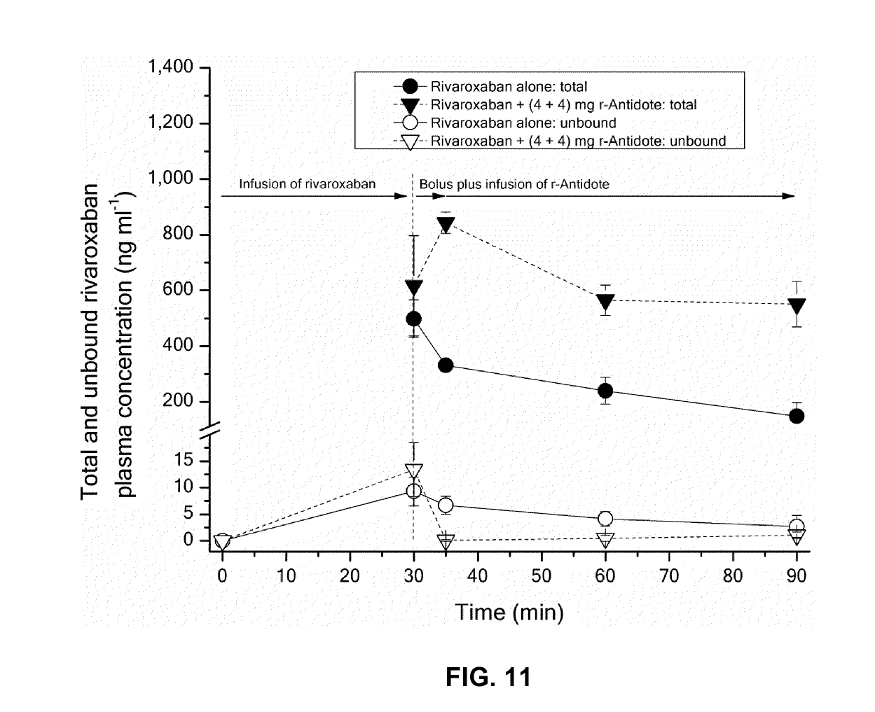

In this example, the ability of r-Antidote to reverse the anticoagulant effect of three direct fXa inhibitors in a rat model was tested. As shown in FIG. 10A, infusion of rivaroxaban (0.25 mg/kg/hr) over a 30 min period produced a 2-fold increase in whole blood INR (International Normalized Ratio). Upon cessation of infusion, coagulation marker in the vehicle treated group decreased gradually due to clearance of circulating inhibitor. Immediately following an IV bolus injection of r-Antidote (4 mg/rat), whole blood INR was rapidly and completely reversed to baseline values, and this reversal was sustained by an infusion of r-Antidote (4 mg/hr). Baseline values in rat blood correspond to an INR of 2.2 rather than 1, since the cartridges used for measurement are calibrated for human use. The effect of r-Antidote administration on pharmacokinetics of rivaroxaban is shown in FIG. 11. Upon intravenous administration of r-Antidote, the total rivaroxaban concentration increased immediately due to redistribution of rivaroxaban from extravascular compartments. Even though there was an increase in total rivaroxaban concentration, the amount of free, non-protein bound rivaroxaban (the fraction responsible for anticoagulant activity) decreased to very low levels. Thus a decrease in free fraction of rivaroxaban was correlated to the correction of whole blood INR. The molar ratio of r-Antidote/rivaroxaban (total) during the experimental time course was 2.1 (35 min), 1.7 (60 min) and 1.3 (90 min) respectively. Similar experiments were carried out using betrixaban and apixaban infusion into anesthetized rats (FIG. 10B and FIG. 10C). As before, serial blood samples were collected to measure whole blood INR, plasma concentration of r-Antidote, total and free fraction of betrixaban and apixaban. Following IV infusion of the three individual fXa inhibitors for 30 min, the total plasma concentrations of rivaroxaban, betrixaban and apixaban were 1.4, 0.2 and 1.4 μM and the % unbound concentrations were 2.2%, 41% and 1.5%, respectively. Upon administration of r-Antidote, total plasma concentration increased to 1.9, 2.0 and 4.2 μM while the % unbound concentrations of the inhibitors declined to 0%, 0.3% and 0.05% of total inhibitor concentration, respectively. Thus, reversal of all three inhibitors followed a common mechanism of reduction of free fraction, which translated to a concomitant correction of PT's to near-normal levels.

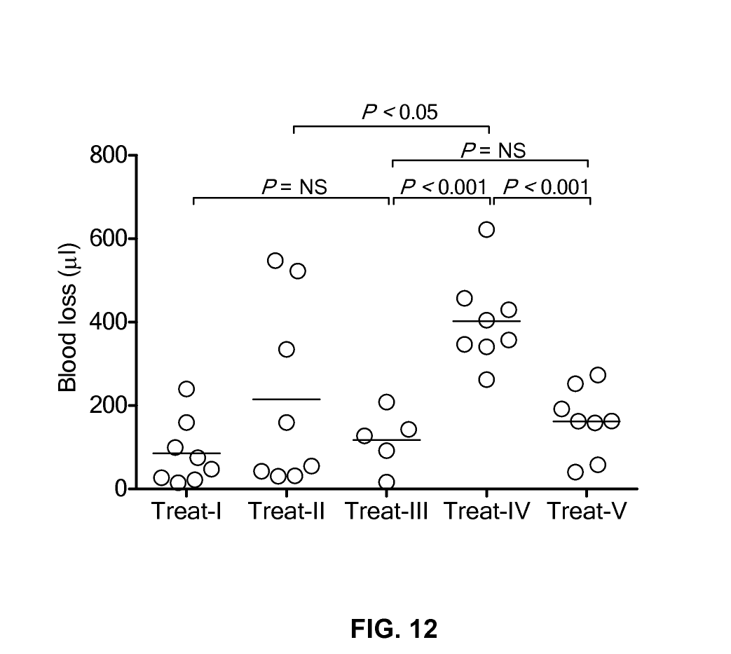

In order to evaluate if reversal of markers of anticoagulation translate into cessation of bleeding, the ability of r-Antidote to restore normal hemostasis in animal models of blood loss was tested. As shown in FIG. 12, oral administration of rivaroxaban alone at a high dose (50 mg/kg) did not produce consistent levels of blood loss in mice (see FIG. 12, Treat-II, blood loss=216±222 μL, mean±SD). Compared to vehicle controls (Treat-I; 86±79 μL), aspirin (ASA) monotherapy with oral dose ˜100 mg/kg/day for 5 days did not result in statistically significant increase in blood loss (Treat-III; 118±71 μL). Combination of ASA with a single oral dose of rivaroxaban at 50 mg/kg (Treat-IV, ASA+rivaroxaban+vehicle) increased blood loss by 3.4-fold (403±107 μL) compared to ASA alone. An IV injection of r-Antidote (Treat-V, 0.96 mg r-Antidote/mouse), 2 hours post oral dose of rivaroxaban, reduced the increased blood loss by ˜84% (163±82 μL) relative to rivaroxaban and ASA background.

The effect of r-Antidote on circulating rivaroxaban concentration was similar to previous observations in the rat. Following r-Antidote administration, total rivaroxaban concentration in plasma was increased by 7.8-fold (Treat-V), while in parallel to correction of blood loss, rivaroxaban anticoagulant activity was reduced by>80% as measured by anti-fXa activity (data not shown).

In this example, rats (male, SD, Charles River) were anesthetized with intraperitoneal administration of ketamine cocktail and a jugular and femoral vein catheterized for fXa inhibitor and r-Antidote administration and serial blood sampling. Blood sampling catheter patency was maintained by slow infusion of normal saline. Serial blood samples were obtained during the experimental time course. Total fXa inhibitor concentration was measured by HPLC with tandem mass spectrometry. The free fraction of fXa inhibitor, not bound to plasma proteins was determined by ultra-filtration using a Microcon device, followed by HPLC-MS/MS quantitation. r-Antidote concentration was measured by ELISA as described previously.

For the mouse tail transection blood loss model, mice (male, C57 BL/6, Charles River) were first treated with aspirin at ˜100 mg/kg/day for 5 days via drinking water. A single, oral administration of rivaroxaban (50 mg/kg) or vehicle (0.5 % methylcellulose) increased blood loss 3.4-fold compared to ASA group. Mice were anesthetized and r-Antidote (0.96 mg/mouse, IV bolus) or vehicle (formulation buffer, 200 μL/mouse) was administered 2 hours after the oral dose of rivaroxaban and prophylactically prior to initiation of blood loss (15 min by tail transection similar to the rat model). Cardiac blood samples at the end of experiment were used for measurements of rivaroxaban, r-Antidote plasma concentrations, whole blood INR and anti-fXa activity.

### Example 8

### r-Antidote Restores Hemostasis in Rabbits Anticoagulated with fXa Inhibitor

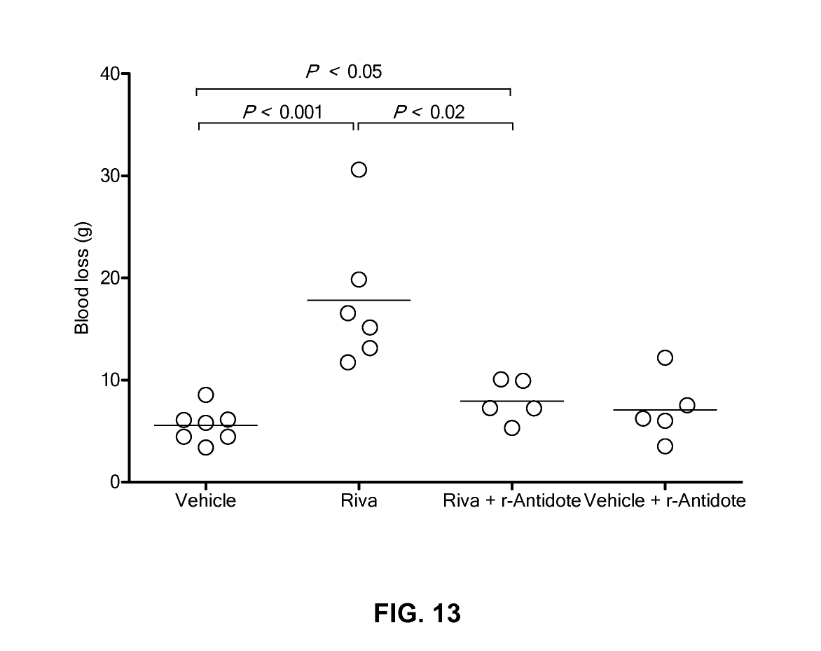

Since tail transection in mice or rats may not be representative of anticoagulant induced bleeding after major trauma and fXa inhibitors are often used as monotherapy in the absence of aspirin, the potential for restoration of hemostasis by r-Antidote in a rabbit model of liver laceration was further investigated. Anesthetized rabbits were administered vehicle or 1 mg/kg rivaroxaban via IV bolus injection. After 30 minutes to allow biodistribution for rivaroxaban, vehicle or r-Antidote (75 mg/rabbit) was administered as an injection followed by laceration of two liver lobes and lost blood was collected on pre-weighed gauze over 15 minutes. Blood loss due to rivaroxaban anticoagulation represented approximately 10% of the animal's total blood volume (FIG. 13). As in the rodent model, total and free rivaroxaban concentration in plasma, anti-fXa activity and PT and activated partial thromboplastin times (aPTT) was measured.

Rivaroxaban dosing produced a circulating concentration of 1.65 μM at 30 min, resulting in a 2.3-fold and 1.9-fold prolongation of PT and aPTT, respectively and increased blood loss by 3.2-fold over vehicle. Administration of r-Antidote reduced blood loss by >85% and decreased peak anti-fXa activity by 98%, PT by 74%, aPTT by 66% and altered the non-protein bound fraction of rivaroxaban in plasma from 26% to <0.5%. r-Antidote alone had no effect on blood loss. Similar to the observations in rat model of reversal of ex vivo clotting parameters (FIG. 10), the rabbit model correlated the correction of rabbit visceral blood loss to a reduction in anti-fXa activity and free fraction of direct fXa inhibitor.

In this example, a modified rabbit (male, NZW, Charles River) liver laceration model was used to demonstrate the effects of r-Antidote to reverse rivaroxaban-induced anticoagulation. Rivaroxaban (1 mg/kg) or vehicle was dosed to anesthetized rabbits by IV bolus over 2 minutes via marginal ear vein catheter. After 30 minutes, r-Antidote or vehicle was administered as a bolus IV injection over 5 minutes via contralateral ear vein catheter followed by laceration of two liver lobes with a scalpel blade (5× each lobe: 1-cm long and 3-mm deep incisions) and lost blood was collected on pre-weighed gauze over 15 minutes. Serial blood samples were collected at 0, 30, 35, and 50 minutes post rivaroxaban. Measurements included reduction in blood loss, decrease in unbound rivaroxaban in the plasma using equilibrium dialysis method, anti-fXa activity, PT and aPTT. The method for measuring PT is described previously in Example 6.

### Example 9

### Reversal of ATIII-Dependent fXa Inhibitors by r-Antidote

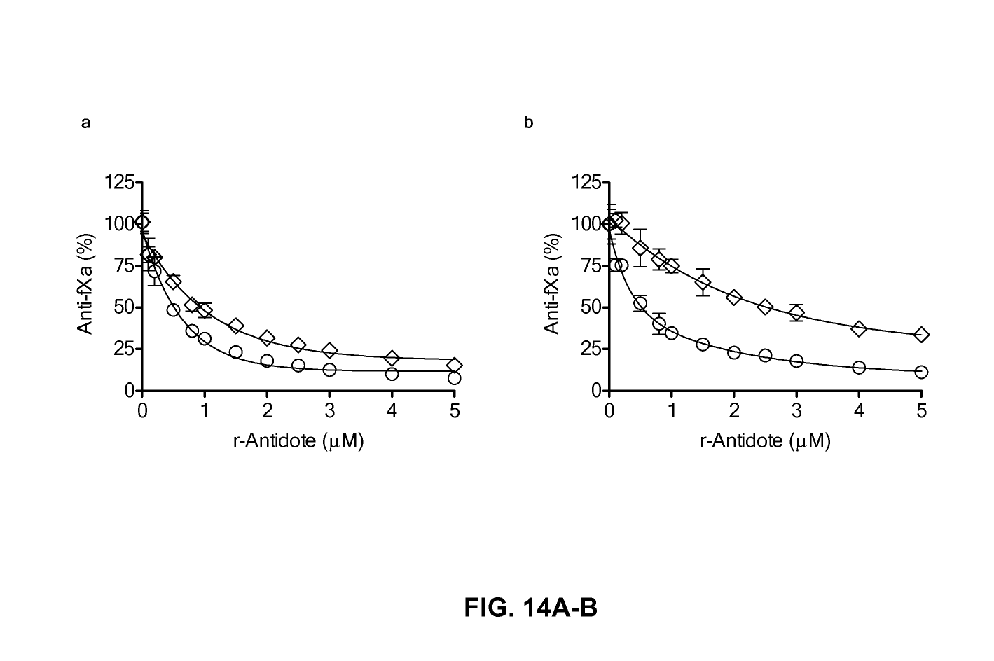

In this example, the effect of r-Antidote on enoxaparin and fondaparinux anticoagulant activity was tested. The anti-fXa activity of therapeutic levels of LMWH (enoxaparin, 1 IU/mL) and fondaparinux (2 μg/mL) was dose-dependently reversed following incubation with r-Antidote in either human or rat plasma (FIG. 14, panels A and B).

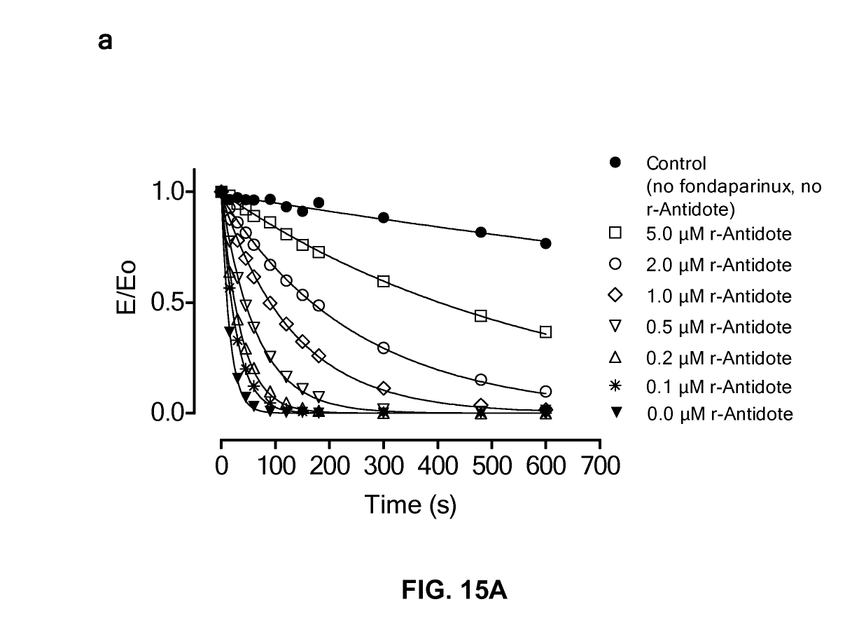

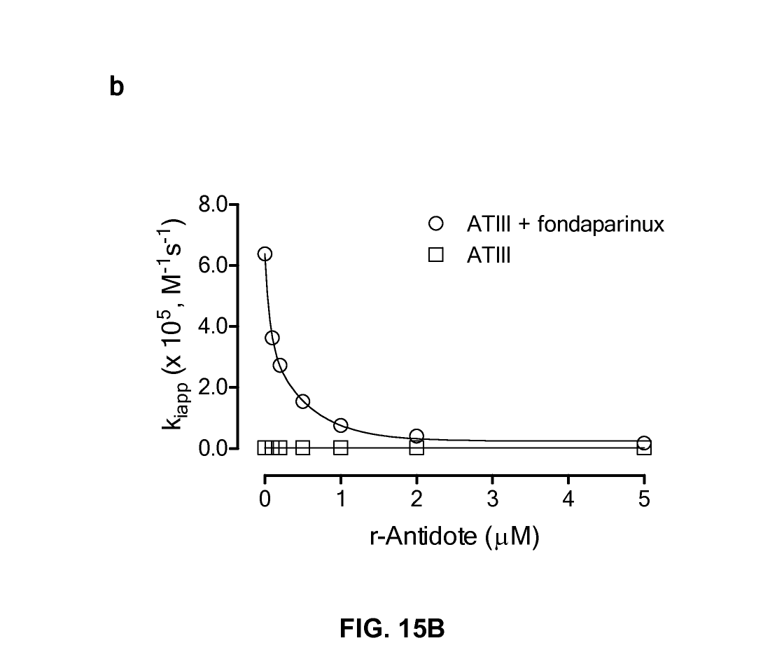

To further investigate the specific interaction between r-Antidote and ATIII, an evaluation was carried out in a reconstituted system with purified human proteins (fXa, ATIII, fondaparinux). FIG. 15A shows reversal of fXa activity in the presence of increasing concentrations of r-Antidote. The solid lines represent the non-linear fitting of the experimental data with a single exponential decay function. Using this fit, an apparent association rate constant (k_{obs}) for the reaction were calculated with different concentrations of r-Antidote. In the absence of fondaparinux, r-Antidote had minimal effect on the inhibition of fXa by ATIII (data not shown). As shown in FIG. 15B, the interaction of r-Antidote with ATIII-fondaparinux complex was different from that observed with ATIII alone. This was due to the different binding affinity of r-Antidote for the ATIII-fondaparinux complex versus ATIII alone. The program Dynafit was used to perform kinetic analyses of curves generated using a series of ATIII-fondaparinux and PRT064445 concentrations. This analysis yielded a dissociation constant of 53 nM for PRT064445/ATIII-fondaparinux complex. Synthetic pentasaccharide has been shown to enhance the binding affinity of ATIII with active-site modified human fXa mutant. By kinetic experiments, the reported dissociation constants range from 120-255 nM and by surface plasmon resonance, a Kd of 610 nM was determined.

Anti-fXa activity was measured using an adapted assay using reagents from a commercial Heparin kit (available from Coamatic, DiaPharma). Pooled platelet poor plasma from healthy human donors or animals (mouse or rat) was prepared in 0.32% citrate (pH=7.0) for generation of standard curves and sample dilution. Bovine fXa and S2732 fXa substrate were reconstituted according to manufacturer's instructions. The reaction mixture contained 75 μL citrated plasma sample or standard, 50 μL substrate S2732. Following pre-incubation at RT for 30 min, 25 μL bovine fXa was added to the mixture and residual fXa activity was determined by measuring cleavage of substrate at RT for 5 min. The reaction was quenched by adding 20% acetic acid (50 μL). The standard curves were constructed with known concentrations of each individual anticoagulant.

Kinetics of fXa inhibition by ATIII and the ATIII-fondaparinux complex were carried out under pseudo-first order reaction conditions where the ATIII concentration exceeded the fXa concentration in the fXa+ATIII reaction. In reactions containing fondaparinux, a limiting amount of fondaparinux was mixed with saturating ATIII concentrations. Both ATIII and fondaparinux concentrations used were over the reported Kd for the complex formation (Kd=32 nM). The reaction mixtures contained human fXa (20 nM), human ATIII (Haematologic Technologies) (200 nM), and fondaparinux (GlaxoSmithKline) (100 nM) along with increasing concentrations of r-Antidote in TBS buffer containing Ca²⁺(5 mM). Following initiation of the reaction, 10 μL of reaction mixture were removed at various time points and assayed for residual fXa activity by measuring Spectrozyme-fXa cleavage. fXa activity was normalized by comparing it to the activity at t=0 before addition of either fondaparinux or ATIII (E/Eo).

### Example 10

### Correction of Blood Loss Due to Enoxaparin and Fondaparinux Anticoagulation in Rats

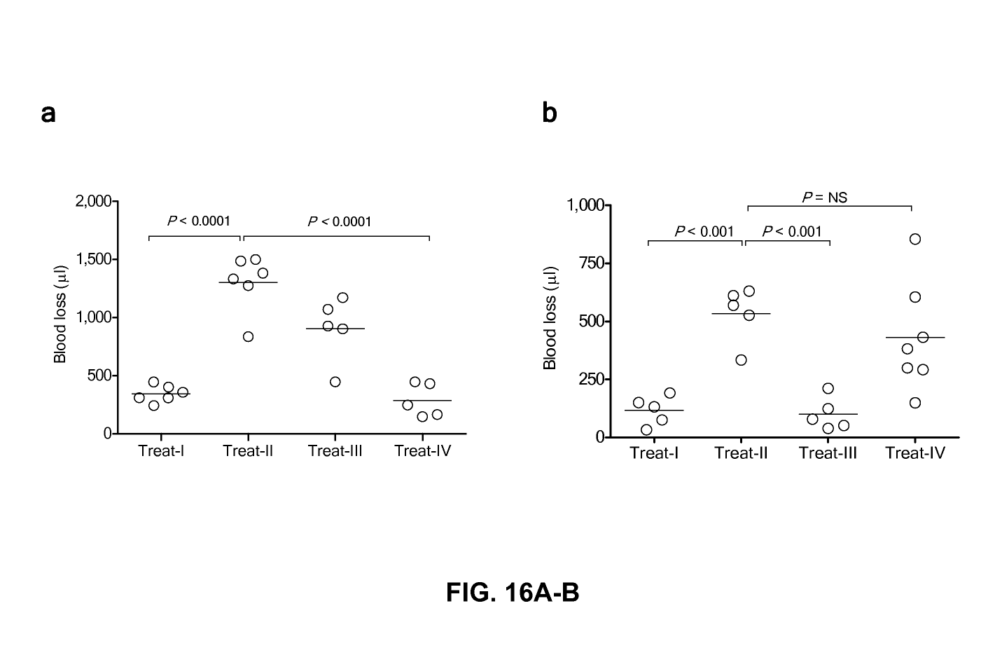

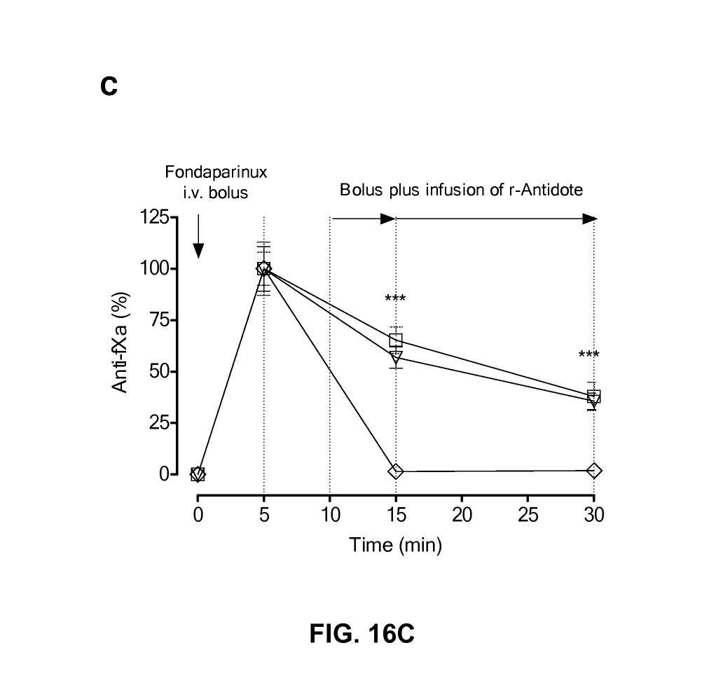

In this example, the ability of r-Antidote to restore hemostasis in a rat tail transection model following treatment with enoxaparin or fondaparinux was tested. Unlike in the case of rivaroxaban, administration of enoxaparin or fondaparinux without concomitant administration of anti-platelet agent was sufficient to establish a reproducible pattern of bleeding in the animals. Two different doses of r-Antidote for reversal of blood loss following the same enoxaparin dose (4.5 mg/kg IV bolus) were evaluated. As shown in FIG. 16A, r-Antidote treatment with 4 mg/rat bolus plus 4 mg/hr infusion completely corrected the increase in blood loss to baseline levels while a lower dose (2 mg/rat bolus plus 2 mg/hr infusion) resulted in a partial (42%) correction in blood loss. In parallel to corrections in blood loss, the anti-fXa activity was reduced in a dose-dependent manner (data not shown).

LMWHs, such as enoxaparin, have both anti-fXa and anti-thrombin inhibitory activity. In order to demonstrate the ability of r-Antidote to reverse fXa specific indirect inhibitors, reversal of fondaparinux anticoagulation in the rat tail transection model were further tested. As shown in FIG. 16B, supra-therapeutic doses of fondaparinux (25 mg/kg) were required to produce a statistically significant increase in blood loss. Administration of r-Antidote (6 mg/rat bolus plus 6 mg/hr infusion) produced a complete cessation of bleeding. In parallel, r-Antidote also substantially reduced the anti-fXa activity of fondaparinux (FIG. 5C). Protamine, at the tested dose (0.9 mg/rat), had no statistically significant effect on fondaparinux anticoagulation (p=0.39).

In this example, a rat tail transection model was used to study the effect of r-Antidote on blood loss induced by anticoagulation with enoxaparin and fondaparinux. Rats were anesthetized and catheterized for serial blood sampling and administration of anticoagulant and r-Antidote. Rats were prophylactically administered r-Antidote prior to initiation of blood loss, subsequently the tail was transected with a scalpel blade and immersed in normal saline at 37° C., followed by a 15 minute r-Antidote infusion and continuous collection of blood. The collected blood samples were processed by freezing the samples at −80° C. to lyse erythrocytes. Hemoglobin concentration and corresponding blood volume were quantified spectrophotometrically (Absorbance at 490nm) from a standard curve constructed with known volumes of blood. Blood samples were collected at 0, 5, 15, 30 min for anti-fXa activity and r-Antidote concentration measurements.

### Example 11

### Randomized, Double-Blind, Placebo-Controlled Single Ascending Dose Pharmacokinetic and Pharmacodynamic Study of r-Antidote

Direct factor Xa (fXa) inhibitors have demonstrated superior anticoagulant efficacy and/or safety relative to older drugs but are limited by the absence of a specific antidote to reverse anticoagulation during episodes of serious bleeding. r-Antidote is a modified, human recombinant fXa that is catalytically inactive but still retains the ability to bind with high affinity direct fXa inhibitors as well as heparin-antithrombin III complexes. In both cases, it competes with native fXa for the inhibitor drugs, thus reversing direct and indirect fXa inhibitor-mediated anticoagulation.

In this example, 32 healthy volunteers were randomized (6:2) within each dosing cohort to a single IV bolus of r-Antidote given in ascending doses of 30 mg, 90 mg, 300 mg, and 600 mg or placebo in a double-blind manner and followed for 28 days. Anti-fXa activity was assayed in vitro by adding exogenous rivaroxaban (50 ng/mL) to subject plasma samples.

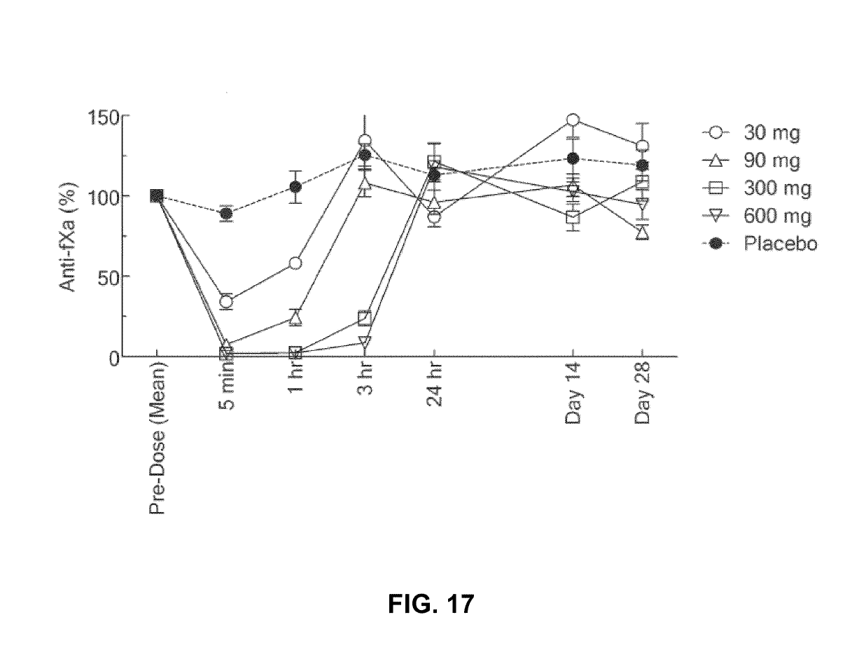

The terminal t1/2 was ˜6 hours. AUC and Cₘₐₓ increased disproportionately relative to dose. Cₘₐₓ was 2.2 μM at the 600 mg dose. In the presence of r-Antidote, thrombin generation and anti-fXa activity of rivaroxaban were reversed in a dose-dependent manner (FIG. 17). There were no thrombotic adverse events or deaths. There was 1 serious adverse event (pneumonia, 30 mg) and 3 non-serious infusion-related reactions without anaphylaxis [90 mg (2) and 300 mg (1)]. One unplanned pregnancy occurred ˜10 days post-treatment (600 mg), followed shortly by a spontaneous abortion. Prothrombin fragment 1+2, thrombin-antithrombin complex, and D-dimer increased with dose; other coagulation parameters were unremarkable.

This example demonstrates that r-Antidote is a promising universal antidote for fXa inhibitors in humans at the doses tested.

### Example 12

### A Phase 2 Randomized, Double-Blind, Placebo-Controlled Trial of r-Antidote

This example aims to evaluate dose-response of r-Antidote in reversing the anticoagulant effects of several fXa inhibitors, pharmacokinetics (PK), and overall safety in up to 144 healthy volunteers.

The effects of r-Antidote with with apixaban, rivaroxaban, enoxaparin, and betrixaban (collectively FXaI) are being studied. Each fXaI will be studied with up to 4 different dose cohorts of r-Antidote or placebo in a 6:3 ratio (i.e., up to 36 subjects per fXaI). Subjects are treated on Days 1-6 with fXaI (to steady state) and then dosed with IV r-Antidote or placebo on Day 6, 3 hours after the fXaI dose. Pharmacodynamic and safety data are collected through Day 48 and PK through Day 10. A chromogenic assay was used to determine anti-fXa activity.

Available data from the first r-Antidote dose cohort of 9 subjects, who received apixaban 5 mg bid (11 doses), followed by a bolus of r-Antidote (90 mg) or placebo IV shows that anti-fXa activity peaked at 3 hours post last apixaban dose. Two minutes after r-Antidote mean anti-fXa activity decreased by −65% (n=4) vs. +6% in the placebo group (n=2). At 10, 30, and 60 minutes post-r-Antidote or placebo, the comparable values were −47% vs. +7%; −29% vs. +11%; and −17% vs. +3%. Similarly, plasma concentrations of unbound apixaban decreased immediately after r-Antidote but were unchanged following placebo. Based on a mean apixaban plasma concentration of 474 nM and an estimated r-Antidote plasma concentration of 314 nM, the r-Antidote/apixaban molar ratio at a dose of 90 mg was 0.66, indicating that this initial low dose was able to bind approximately two thirds of the apixaban in the plasma. This is in good agreement with the observed 65% decrease in anti-fXa activity. r-Antidote was temporally associated with transient reduction in tissue factor pathway inhibitor (TFPI) and increase in F1+F2, but no change in D-dimer. There were no thrombotic or allergic-type adverse events, serious adverse events, or deaths. r-Antidote was well tolerated; 5/9 subjects experienced one or more adverse events; all were mild.

In conclusion, emerging in vivo data confirms prior ex-vivo observations that r-Antidote reverses anticoagulant effects of fXaI. r-Antidote is a well-tolerated, and potentially promising universal antidote for fXaI.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A unit dose formulation for neutralizing a factor Xa inhibitor, comprising a pharmaceutically acceptable carrier and from about 25 milligrams to about 95 milligrams of a two-chain polypeptide comprising the amino acid sequence of SEQ ID NO. 3 or a polypeptide having at least 80% sequence identity to SEQ ID NO. 3, wherein the polypeptide that has at least 80% sequence identity to SEQ ID NO. 3 (a) has reduced procoagulant activity compared to wild-type factor Xa and (b) does not assemble into a prothrombinase complex.

2. The unit dose formulation of claim 1, having from about 30 milligrams to about 80 milligrams of the polypeptide.

3. The unit dose formulation of claim 1, having from about 35 milligrams to about 70 milligram of the polypeptide.

4. The unit dose formulation of claim 1, having from about 35 milligrams to about 60 milligrams of the polypeptide.

5. The unit dose formulation of any preceding claim, formulated for administration as a single bolus.

6. The unit dose formulation of any preceding claim wherein the polypeptide is lyophilized.

7. The unit dose formulation of one of claims 1-5, wherein the carrier is saline.

8. A container comprising a pharmaceutically acceptable carrier and from about 25 milligrams to about 95 milligrams of a two-chain polypeptide comprising the amino acid sequence of SEQ ID NO. 3.

9. A method of selectively binding and inhibiting an exogenously administered direct factor Xa inhibitor in a subject undergoing anticoagulant therapy with a direct factor Xa inhibitor comprising administering to the subject an injection of a unit dose formulation of claim 1.

10. A method of preventing, reducing, or ceasing bleeding in a subject undergoing anticoagulant therapy with a direct factor Xa inhibitor comprising administering to the subject an injection of a unit dose formulation of claim 1.

11. A method for correcting fXa inhibitor-dependent pharmacodynamic or surrogate markers in a patient undergoing anticoagulant therapy with a direct factor Xa inhibitor comprising administering to the subject an injection of a unit dose formulation of claim 1.

12. The method of claim 11, wherein the pharmacodynamic or surrogate marker is selected from the group consisting of INR, PT, aPTT, ACT, anti-fXa units, and thrombin generation.

13. The method of any one of claims 10-12, wherein the direct factor Xa inhibitor is selected from the group consisting of NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

14. The method of claim 13, wherein the direct factor Xa inhibitor is betrixaban.

15. The method of claim 13, wherein the direct factor Xa inhibitor is rivaroxaban.

16. The method of claim 13, wherein the direct factor Xa inhibitor is apixaban.

17. A method of selectively binding and inhibiting an exogenously administered indirect factor Xa inhibitor in a subject undergoing anticoagulant therapy with an indirect factor Xa inhibitor comprising administering to the subject an injection of a unit dose formulation of claim 1.

18. A method of preventing, reducing, or ceasing bleeding in a subject undergoing anticoagulant therapy with an indirect factor Xa inhibitor comprising administering to the subject an injection of a unit dose formulation of claim 1.

19. A method for correcting fXa inhibitor-dependent pharmacodynamic or surrogate markers in a patient undergoing anticoagulant therapy with an indirect factor Xa inhibitor comprising administering to the subject an injection of a unit dose formulation of claim 1.

20. The method of any one of claims 17-19, wherein the indirect factor Xa inhibitor is selected from the group consisting of fondaparinux, idraparinux, biotinylated idraparinux, enoxaparin, fragmin, tinzaparin, low molecular weight heparin and combinations thereof.

21. The method of claim 20, wherein the indirect factor Xa inhibitor is enoxaparin.

22. A method of reducing or ceasing bleeding in a subject undergoing an anticoagulant therapy with a direct factor Xa inhibitor, comprising administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reaches a circulating molar concentration in the subject that is less than about 95% of the circulating molar concentration of the direct factor Xa inhibitor, wherein the polypeptide that has at least 80% sequence identity to SEQ ID NO. 3 (a) has reduced procoagulant activity compared to wild-type factor Xa and (b) does not assemble into a prothrombinase complex.

23. The method of claim 22, wherein the polypeptide reaches a circulating molar concentration in the subject that is less than about 70% of the circulating molar concentration of the direct factor Xa inhibitor.

24. The method of claim 22 or 23, wherein the direct factor Xa inhibitor is selected from the group consisting of NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

25. The method of claim 24, wherein the direct factor Xa inhibitor is betrixaban.

26. The method of claim 24, wherein the direct factor Xa inhibitor is rivaroxaban.

27. The method of claim 24, wherein the direct factor Xa inhibitor is apixaban.

28. A method of reducing or ceasing bleeding in a subject undergoing an anticoagulant therapy with an indirect factor Xa inhibitor, comprising administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reduces from about 20% to about 95% of the level of an anti-fXa pharmacodynamic marker of the indirect factor Xa inhibitor, wherein the polypeptide that has at least 80% sequence identity to SEQ ID NO. 3 (a) has reduced procoagulant activity compared to wild-type factor Xa and (b) does not assemble into a prothrombinase complex.

29. The method of claim 28, wherein the polypeptide reduces from about 25% to about 80% of the level of the anti-fXa pharmacodynamic marker.

30. The method of claim 28, wherein the polypeptide reduces from about 35% to about 65% of the level of the anti-fXa pharmacodynamic marker.

31. The method of any one of claims 28-30, wherein the indirect factor Xa inhibitor is selected from the group consisting of NAP-5, rNAPc2, tissue factor pathway inhibitor, DX-9065a, YM-60828, YM-150, apixaban, rivaroxaban, TAK-442, PD-348292, otamixaban, edoxaban, LY517717, GSK913893, razaxaban, betrixaban or a pharmaceutically acceptable salt thereof, and combinations thereof.

32. The method of claim 31, wherein the indirect factor Xa inhibitor is enoxaparin.

33. The method of any one of claims 28-32, wherein the administration is via a single bolus.

34. A method of reducing or ceasing bleeding in a subject having received an anticoagulant therapy with a factor Xa inhibitor and experiencing clinically relevant bleeding, comprising administering to the subject a therapeutically effective amount of a formulation comprising a two-chain polypeptide comprising the amino acid sequence of SEQ ID NO. 3 or a polypeptide having at least 80% sequence identity to SEQ ID NO. 3.

35. The method of claim 34, wherein the administration comprises an injection of the formulation.

36. The method of claim 34 or 35, wherein the administration is at least about 5 minutes after the blood loss has initiated.

37. The method of any one of claims 34-36, wherein the polypeptide neutralizes between about 20% and about 95% of the factor Xa inhibitor.

38. The method of any one of claims 34-36, wherein the polypeptide neutralizes between about 35% and about 65% of the factor Xa inhibitor.

39. A method of controllably neutralizing the anticoagulant activity of a factor Xa inhibitor in a subject undergoing an anticoagulant therapy with the factor Xa inhibitor, comprising:
measuring the level of an anti-fXa pharmacodynamic marker of the factor Xa inhibitor, and
administering to the subject a therapeutically effective amount of a formulation comprising a two-chain polypeptide comprising the amino acid sequence of SEQ ID NO. 3 or a polypeptide having at least 80% sequence identity to SEQ ID NO. 3, such that the polypeptide reduces from about 20% to about 95% of the level of the anti-fXa pharmacodynamic marker.

40. The method of claim 39, wherein the polypeptide reduces from about 35% to about 65% of the level of the anti-fXa pharmacodynamic marker.


SEQUENCE LISTING (5 sequences)

SEQ ID NO: 1 - protein, 448 aa, Homo sapiens [sequence omitted]

SEQ ID NO: 2 - protein, 365 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRRKRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 3 - protein, 359 aa, Artificial Sequence
  Description of Artificial Sequence Synthetic polypeptide
ANSFLFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDAGGPHVTRFKDTYFVTGIVSWGEGCARKGKYGIYTKVTAFLKWIDRSMKTRGLPKAKSHAPEVITSSPLK

SEQ ID NO: 4 - protein, 6 aa, Artificial Sequence
  Description of Artificial Sequence Synthetic peptide
RKRRKR

SEQ ID NO: 5 - protein, 3 aa, Artificial Sequence
  Description of Artificial Sequence Synthetic peptide
RKR